(19) United States
(12) Patent Application Publication
(10) Pub. No.: US 2021/0338822 A1
(43) Pub. Date: Nov. 4, 2021

(54) THERAPEUTIC EXOSOMES AND METHOD OF PRODUCING THEM

(71) Applicant: AgeX Therapeutics, Inc., Alameda, CA (US)

(72) Inventors: Dana LaRocca, Alameda, CA (US); Jieun Lee, San Mateo, CA (US); Hal Sternberg, Berkeley, CA (US)

(73) Assignee: AgeX Therapeutics, Inc., Alameda, CA (US)

(21) Appl. No.: 17/156,512

(22) Filed: Jan. 22, 2021

Related U.S. Application Data
(60) Provisional application No. 62/964,590, filed on Jan. 22, 2020.

(51) Int. Cl. A61K 47/46; C12N 15/113

(52) U.S. Cl. CPC A61K 47/46; C12N 2320/32; C12N 2310/141; C12N 15/113

(57) ABSTRACT

The invention provides improved methods, compositions, uses and kits relating to exosomes isolated from cells and therapeutic compositions and methods of using those exosomes. In one embodiment, the exosomes are loaded with one or more molecules to provide a desired therapeutic effect.

### RELATED APPLICATIONS

This application claims benefit under 35 USC § 119(e) of U.S. Provisional Patent Application 62/964,590, filed Jan. 22, 2020.

### FIELD OF THE INVENTION

The field of the invention relates to exosomes isolated from progenitor cells.

### BACKGROUND

Exosomes are believed to contain important signaling molecules that may provide the source of trophic factors responsible for some regenerative benefits seen in cell replacement therapy. As such they would provide an alternative to some cell based therapies that would be easier to manufacture on a large scale and potentially safer to administer to a subject in need of cell therapy. In particular, the risk associated with transmission of infectious agents such as viruses may be lower compared to transplanting whole cells. Moreover, the risk of immune rejection of the exosomes relative to transplanted cells may also be lower. Accordingly, exosomes may provide an attractive alternative or adjunct to cell based therapies and cell based regenerative medicine.

Exosomes are 30 to 120 nm vesicles secreted by a wide range of mammalian cell types. Keller et al. (2006) Immunol Lett. 107(2):102; Camussi et al. (2010) Kidney International 78:838. The vesicles are enclosed by a lipid bilayer and are larger than LDL which has a size of 22 nm, but smaller than a red blood cell, which is 6000 to 8000 nm in diameter and has a thickness of 2000 nm Keller et al. (2006) Immunol Lett. 107(2):102.

Exosomes are found both in cells growing in vitro as well as in vivo. They can be isolated from tissue culture media as well as bodily fluids such as plasma, urine, milk and cerebrospinal fluid. George et al. (1982) Blood 60:834; Martinez et al. (2005) Am J Physiol Health Cir Physiol 288:H1004. Exosomes originate from the endosomal membrane compartment. They are stored in intraluminal vesicles within multivesicular bodies of the late endosome. Multivesicular bodies are derived from the early endosome compartment and contain within them smaller vesicular bodies that include exosomes. Exosomes are released from the cell when multivesicular bodies fuse with the plasma membrane. See FIG. 1. Methods of isolating exosomes from cells has been described, see e.g. US Patent Application Publication No. 20120093885.

Exosomes contain a variety of molecules including proteins, lipids and nucleic acids such as DNA, mRNA and miRNA. Their contents are believed to play a part in cell to cell communication involving the release of the exosome from one cell and the binding/fusion of the exosome with a second cell, wherein the contents of the exosomal compartment are released within the second cell.

It has been reported that exosomes derived from endothelial progenitor cells may act as vehicle for mRNA transport among cells. Once incorporated into the endothelial cells, the exosomes stimulated an angiogenic program. Deregibus et al. (2007) Blood 110:2440. Similar results were obtained in vivo using severe combined immunodeficient mice. Exosome stimulated endothelial cells implanted subcutaneously in Matrigel (a murine sarcoma extract) organized into a patent vessel network connected with the murine vasculature. Deregibus, supra. Bruno et al. (2009) J Am Soc Nephrol 20:1053; Herrera et al. (2010) J Cell Mol Med 14:1605.

Of the various molecular cargo of exosomes, miRNAs have attracted attention due to their regulatory roles in gene expression. MiRNAs are small, non-coding regulatory RNAs that can have a wide range of effects on multiple RNA targets, thus having the potential to have greater phenotypic influence than coding RNAs. MiRNA profiles of exosomes often differ from those of the parent cells. Profiling studies have demonstrated that miRNAs are not randomly incorporated into exosomes but rather a subset of miRNAs is preferentially packaged into exosomes, suggesting an active sorting mechanism of exosomal miRNAs. Guduric-Fuchs et al. (2014) Nucleic Acid Res. 42:9195; Ohshima et al. (2010) PloS One 5(10):e13247.

Certain isolated exosomes, methods for their production, and their characterization have been published. See, e.g., U.S. Pat. No. 10,240,127.

Nevertheless, there remains a need for improved exosome compositions, methods of producing those exosome compositions, and therapeutic uses of exosome compositions.

### SUMMARY OF THE INVENTION

In various embodiments described herein the invention provides compositions comprising exosomes obtained from progenitor cell lines, as well as methods of making and using exosomes obtained from progenitor cell lines. For example, the invention may involve exosomes isolated from progenitor cell lines 30-MV2-14, 30-MV2-4, E69 or RPI-MV2-8.

The isolation of embryonic progenitor cells has been described. See West et al. (2008) Regen Med 3:287; US Patent Application Publication Nos. 20080070303 20100184033; U.S. Pat. No. 10,240,127.

The present invention is directed to improved methods of preparing exosomes, loaded exosome compositions, and therapeutic uses for exosomes according to the invention.

Exosomes according to the invention may be isolated from cell lines derived under a variety of culture conditions from pluripotent stem cells, such as human embryonic stem (hES) cells or induced pluripotent stem (iPS) cells. The progenitor cell lines are clonal and while they do, in most instances, senesce, they also possess longer telomeres compared to adult or fetal derived tissue or cells (such as adult stem cells) and accordingly have enhanced replicative capacity relative to those cell types. Because of their clonality and their enhanced replicative capacity they provide a suitable source of exosomes that will offer the benefit of uniformity with regard to the exosome composition and abundance relative to exosomes derived from their typical sources such as adult cells or adult stem cells.

In certain embodiments the invention provides an exosome isolated from a progenitor cell line, such as clonal progenitor cell line. In a preferred embodiment, the clonal progenitor cell line is 30-MV2-14, 30-MV2-4, E69 or RPI-MV2-8.

In certain embodiments the invention provides an exosome isolated from a human progenitor cell line, such as a clonal human progenitor cell line.

In some embodiments the invention provides an exosome isolated from endothelial progenitor cell.

In some embodiments the invention provides an exosome isolated from a clonal human endothelial progenitor cell.

In some embodiments, one or more exosomes is loaded with one or more molecules, preferably producing one or more exosomes that are capable of providing a therapeutic effect.

In one embodiment, exosomes according to the invention are capable of healing or accelerating the healing of a wound.

In another embodiment exosomes according to the invention are capable of promoting or accelerating angiogenesis.

In another embodiment exosomes according to the invention are capable of promoting or accelerating epigenetic rejuvenation.

In another embodiment, exosomes according to the invention are capable of altering senolytic activity.

In another embodiment, exosomes according to the invention are capable of cardiac repair or regeneration.

In another embodiment, exosomes according to the invention are capable of cardioprotection.

In another embodiment, exosomes according to the invention are capable of neuroprotection.

In another embodiment, exosomes according to the invention are capable of reducing, slowing, or eliminating the effects of aging.

In another embodiment, exosomes according to the invention are capable of regulating immune activity.

In another embodiment, exosomes according to the invention are capable of enhancing vaccination outcome or vaccination potency.

In another embodiment, exosomes according to the invention are capable of effecting regeneration or repair of endoderm derived tissues, regeneration or repair of endochondral bone formation, chondrocyte differentiation, immunological function (preventing or treating infectious disease, autoimmune disease, allergy, or vaccine potency), leukocyte migration, inflammatory response, inflammation effector, healing (e.g., following injury, trauma, ischemic event), antimicrobial effect, antigen processing and presentation, platelet activation, cardioprotective inflammation effector, regulate immune activity, and skin protection.

In another embodiment, the invention provides an improved process for producing exosomes.

### BRIEF DESCRIPTION OF DRAWINGS

For a fuller understanding of the nature and advantages of the present invention, reference should be had to the following detailed description taken in connection with the accompanying drawings.

FIG. 1 depicts the natural biogenesis of exosomes in a secreting cell and their targeting in a recipient cell.

FIG. 2 is a graph showing lack of MHC antigens in PureStem exosomes demonstrating a lower risk of immune response.

FIG. 3 is a graph showing relative wound density (%) over time in a wound healing assay and images of those cells (with added exosomes and exosome-free) at 0 and 14 hours.

FIG. 4 is a graph showing relative wound density (%) over time in a wound healing assay and images of those cells (with added exosomes and exosome-free) at 0 and 14 hours.

FIG. 5 is a graph showing relative wound density (%) over time in a wound healing assay and images of those cells (with added exosomes and exosome-free) at 0 and 14 hours.

FIG. 6 shows selection of angiogenic PureStem exosomes.

FIG. 7 shows selection of angiogenic PureStem exosomes.

FIG. 8 shows selection of angiogenic PureStem exosomes.

FIG. 9 shows selection of angiogenic PureStem exosomes.

FIG. 10 shows selection of angiogenic PureStem exosomes and how strong wound healing correlates with angiogenic activity.

FIG. 11 shows the diversity of cells and PureStem transcriptomics.

FIG. 12 shows PureStem exosome RNA cargo content, including angiogenic miRNAs and mRNAs.

FIG. 13 shows the stable production of embryonic progenitor exosomes.

FIG. 14 shows a graph of relative wound density (%) over time, showing an example of miRNA loaded exosomes with an increase in wound healing activity over exosome free or scrambled miRNA loaded exosomes.

FIG. 15 is a table of data showing exosomes derived from 30-MV2-4, 30-MV2-14 and RP1-MV2-8 induce functional antiogenesis and that strong wound healing activity of PureStem exosomes correlates with angiogenic activity.

FIG. 16 is a table showing production yield and purity of exosomes isolated from cell lines and 30-MV2-14, 30-MV2-14, RP1-MV2-8 according to the TFF-SEC exosome isolation method according to the invention.

FIG. 17 is a table of miRNS contained in PureStem-exosomes and their function.

FIGS. 18A-D is a table of exosomal protein utilities.

FIG. 19 is a table of RP1-MV2-8 exosome miRNA target genes.

FIG. 20 is a table of 30-MV2-4 exosome miRNA target genes.

FIG. 21 is a table of 30-MV2-14 exosome miRNA target genes.

FIG. 22A-E is a table of miRNAs that are enriched in angiogenic exosomes relative to non-angiogenic exosomes.

FIG. 23A-E is RNAseq RPMI values for four progenitor derived exosomes.

FIG. 24 is a list of miRNAs from 4 PureStem exosome lines RP1-MV2-8, E69, 30MV2-4, and 30MV2-14.

FIG. 25 is a table of miRNAs and their roles in wound healing and angiogenesis.

FIGS. 26 A-H are tables of miRNAs and their roles.

FIG. 27 is a depiction of miRNA and wound healing.

FIG. 28 is a depiction of the role of miRNA in angiogenesis.

FIG. 29 is a depiction of miRNAs and their role in aging.

FIG. 30 is a depiction of miRNAs and their roles in aging.

FIGS. 31A-E is a table of protein total abundance for RP1-MV2-8, E-69, 30-MV2-14 and 30-MV2-4.

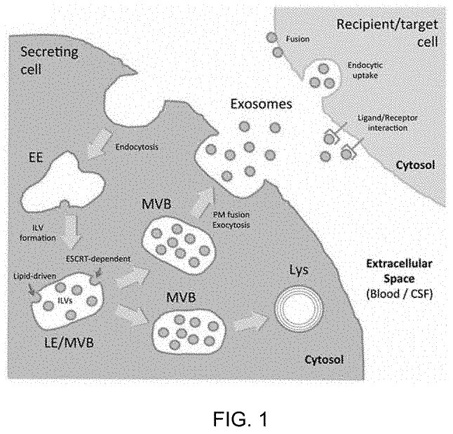

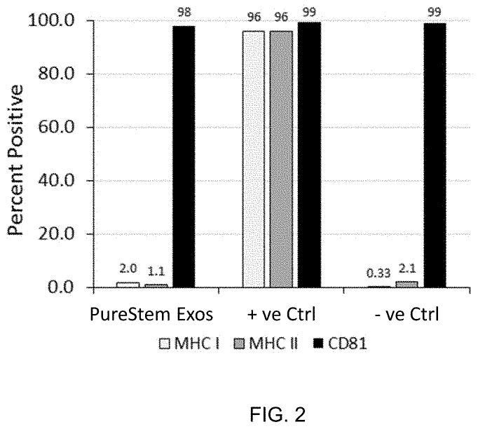

### DETAILED DESCRIPTION

Before the present compositions and methods are described, it is to be understood that this invention is not limited to the particular processes, compositions, or methodologies described, as these may vary. It is also to be understood that the terminology used in the description is for the purpose of describing the particular versions or embodiments only, and is not intended to limit the scope of the present invention which will be limited only by the appended claims. Unless defined otherwise, all technical and scientific terms used herein have the same meanings as commonly understood by one of ordinary skill in the art. Any methods and materials similar or equivalent to those described herein can be used in the practice or testing of embodiments of the present disclosure.

Definitions

As used herein, the singular forms “a,” “an,” and “the” include plural reference unless the context clearly dictates otherwise. Thus, for example, reference to a “therapeutic” is a reference to one or more therapeutics and equivalents thereof known to those skilled in the art, and so forth.

As used herein, the term “about” means plus or minus 10% of the numerical value of the number with which it is being used. Therefore, about 50% means in the range of 45% to 55%.

As used herein, the term “clonal” refers to a population of cells obtained by the expansion of a single cell into a population of cells all derived from that original single cell and not containing other cells. The terms “clonal progenitor cell”, “embryonic clonal progenitor cell”, “clonal progenitor cell line” and “embryonic clonal progenitor cell line” each refer to progenitor cell lines that are derived clonally, i.e., derived by the expansion of a single cell into a population of cells all derived from that original single cell and not containing other cells.

The term “embryonic stem cell” as used herein refers to a pluripotent cell that is derived from a blastocysts, such as an in vitro fertilized blastocyst. Embryonic stem cells include human embryonic stem cells, which are available as established cell lines. The established cell lines are available commercially from numerous public cell banks, e.g. WiCell and private corporations, e.g. ESI BIO.

The term “human pluripotent cell” or “human pluripotent stem cell” as used herein refers to a human cell which is capable of differentiating into at least one cell type found in or derived from each of the three primary germ layers. Some human pluripotent stein cells have the ability to differentiate into all cells found in or derived from each of the three primary germ layers. Examples of human pluripotent stem cells include human embryonic stem cells (Thomson (1998) Science 282:1145), human embryonic germ cells (Shamblott et al. (2001) PNAS 98:113 and induced pluripotent cells (Takahashi et al. (2007) Cell 131:861.

The term “induced pluripotent stem cell” as used herein, refers to a pluripotent cell that has been genetically reprogrammed using any technique known in the art from an adult somatic cell back to the developmentally less mature pluripotent state.

The term “miRNA,” as used herein, refers to microRNA which includes RNA species that are 21-25 nt long and may be single- or double-stranded. MicroRNAs are short, non-coding RNA. molecules that have been found in animals, including humans, and in plants. The term encompasses small interfering RNA (siRNA) and small temporal RNA (stRNA), as well as miRNA proper. miRNAs are transcribed as parts of longer RNA molecules and processed in the nucleus by the dsRNA ribonuclease Drosha to hairpin structures 70-100 nucleotides long. These are transported to the cytoplasm where they are digested to 21-23-mers by the dsRNA ribonuclease Dicer. Single-stranded miRNAs bind to complementary sequences in mRNA thereby inhibiting translation.

“miR-126” is a human microRNA that is specifically expressed in endothelial cells, throughout capillaries and in larger blood vessels. miR-126 plays a role in angiogenesis by regulating the expression levels of various genes by pre- and post-transcription mechanisms. As used herein, the term “miR-126” refers to all of the following: the stem-loop miR-126, miR-126-3p (3′ arm of the hairpin precursor) and miR-126-5p (5′ arm of the hairpin precursor). miRNA naming conventions are described in Kozomara and Griffiths-Jones, (2014) Nucleic Acids Res. 42 (Database issue):D68. The terms “ma-126-3p” and “hsa-miR-126-3p” are also used interchangeably throughout this application.

The use of “nucleic acid,” “polynucleotide” or “oligonucleotide” or equivalents herein means at least two nucleotides covalently linked together. In some embodiments, an oligonucleotide is an oligomer of 6, 8, 10, 12, 20, 30 or up to 100 nucleotides. In some embodiments, an oligonucleotide is an oligomer of at least 6, 8, 10, 12, 20, 30, 40, 50, 60, 70, 80, 90, 100, 150, 200, 300, 400, or 500 nucleotides. A “polynucleotide” or “oligonucleotide” may comprise DNA, RNA, cDNA, PNA or a polymer of nucleotides linked by phosphodiester and/or any alternate bonds.

The term “peptide,” as used herein, refers to two or more amino acids joined by a peptide bond. A peptide can, in some instances, be a portion of a full length protein.

The term “protein” as used herein, refers to a full length protein, i.e. one having all of the amino acids coded for by the mRNA that encodes the particular protein. Also included in the definition are modified proteins where one or more amino acids have been cleaved (e.g. a signal sequence) as a result of the protein being secreted from a cell.

By “pharmaceutically acceptable”, it is meant the carrier, diluent or excipient must be compatible with the other ingredients of the formulation and not deleterious to the recipient thereof.

The term “pluripotent cell” or “pluripotent stem cell” as used herein, refers to a cell which is capable of differentiating into at least one cell type found in or derived from each of the three primary germ layers. Some pluripotent stem cells have the ability to differentiate into all cells found in or derived from each of the three primary germ layers.

The term “progenitor cell line” as used herein refers to a line of cells that is more differentiated (developed) compared to a pluripotent cell, such as iPS cell or an hES cell, but is not terminally differentiated. Progenitor cells will have enhanced replicative capacity compared to a terminally differentiated cell which typically has senesced. Progenitor cells may also have longer telomere lengths compared to a cell that has terminally differentiated. Progenitor cell lines, when cultured, may be able double in population size at least 5, at least 10, at least 20, at least 30, at least 40, at least 50 times. In some instances progenitor cell lines may be able to double in population size 5-400 times, 10-300 times, 20-200 times, 30-80 times, 40-60 times. One example of a progenitor cell line is an embryonic progenitor cell. Embryonic progenitor cell is obtained from a pluripotent cell such as an iPS cell or a hES as previously described. See West et al. (2008) Regen Med 3:287; US Patent Application Publication Nos. 20080070303 20100184033.

The term “subject,” as used herein includes, but is not limited to, humans, non-human primates and non-human vertebrates such as wild, domestic and farm animals including any mammal, such as cats, dogs, cows, sheep, pigs, horses, rabbits, rodents such as mice and rats. In some embodiments, the term “subject,” refers to a male. In some embodiments, the term “subject,” refers to a female.

The term “suitable media,” as used herein, refers to a solution that can be used to grow cells in culture. A suitable media may include a formulation of salts and/or buffering reagents. A suitable media may include any or all of the following: salts, sugars, amino acids, proteins, growth factors, cytokines, and hormones, additives such as serum, albumin, antibiotics, insulin, selenium and transferrin. Suitable culture media includes for example commercially available culture media such as DMEM, MEM Stem Pro and the like.

A “therapeutically effective amount” of a composition such as a therapeutic agent described infra, e.g. an exosome, is a predetermined amount calculated to achieve the desired effect. In some embodiments, the effective amount is a prophylactic amount. In some embodiments, the effective amount is an amount used to medically treat the disease or condition. The specific dose of a composition administered according to this invention to obtain therapeutic and/or prophylactic effects will, of course, be determined by the particular circumstances surrounding the case, including, for example, the composition administered, the route of administration, and the condition being treated. It will be understood that the effective amount administered will be determined by the physician in the light of the relevant circumstances including the condition to be treated, the choice of composition to be administered, and the chosen route of administration. A therapeutically effective amount of composition of this invention is typically an amount such that when it is administered in a physiologically tolerable excipient composition, it is sufficient to achieve an effective systemic concentration or local concentration in the targeted tissue.

The terms “treat,” “treated,” or “treating,” as used herein, can refer to both therapeutic treatment or prophylactic or preventative measures, wherein the object is to prevent or slow down (lessen) an undesired physiological condition, symptom, disorder or disease, or to obtain beneficial or desired clinical results. In some embodiments, the term may refer to both treating and preventing. For the purposes of this disclosure, beneficial or desired clinical results may include, but are not limited to one or more of the following: alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response. Treatment also includes prolonging survival as compared to expected survival if not receiving treatment.

Exosomes of the invention are double membrane bound vesicles secreted from cells of plants and animals, such as mammals including humans, non-human primates, dogs, cats, sheep, cows, pigs, horses, rabbits, mice, rats and guinea pigs to name but a few. Thus exosomes may be isolated from any cell type from any source. In some embodiments of the invention the exosomes of the invention may be secreted from a human cell, such as a human clonal progenitor cell. In some embodiments the exosomes may be secreted from an endothelial human clonal progenitor cell.

Where the exosomes are derived from a clonal progenitor cell, the exosomes will preferably be of uniform quality and composition. Thus, the exosomes isolated from a clonal progenitor cell will not vary as a result of genetic variation of the source cell. The molecular composition of the contents and the bio-physical characteristics of the vesicles will be consistent and reproducible. Moreover, because of the replicative capacity of the human embryonic progenitor cells, the invention provides an overabundance of the exosomes of the invention. This is in direct contrast with exosomes obtained from other sources known in the art where the paucity of the cell type or the problem of senescence limits the availability of a reproducible exosome. Moreover, in certain embodiments the cells giving rise to the exosomes of the invention, are neither transformed nor malignant, thus avoiding any possible concern regarding carcinogenesis of the exosomes.

The exosomes of the invention may have diameter ranging from about 20 nm-130 nm; from about 30 nm-120 nm; about 40 nm-110 nm; about 50 nm-100 nm; about 85 nm-95 nm. In some embodiments the exosomes of the invention have a diameter of about 90 nm. In some embodiments the exosomes of the invention have a diameter of about 88 nm.

The exosomes may be comprised of a lipid bilayer containing trans-membrane proteins and may contain hydrophilic components within the vesicle of the exosome. The contents of the vesicle may be derived from the cytoplasm of the cell or from other vesicle structures within the cell, e.g., endosomes. The vesicle may contain nucleic acids, such as DNA, RNA including mRNA, miRNA as well as proteins and peptides.

The exosomes of the invention may serve as depots for the delivery of therapeutic molecules of any kind. The exosomes of the invention can be engineered to contain therapeutic molecules such as nucleic acids, proteins, peptides, small molecules such as drugs and the like. Any technique known in the art can be used to load the exosomes of the invention with a desired therapeutic molecule. For example cationic lipids could be used to transfect the exosomes with a desired nucleic acid such as DNA, RNA, include mRNA and miRNA. HIV that protein could be used to transport protein or peptide therapeutics into the exosomes of the invention. The therapeutic molecules can be chosen, engineered or designed to have any desired therapeutic effect. For example molecules associated with enhanced angiogenesis could be loaded into the exosomes of the invention, e.g. VEGF.

The secreted exosomes of the invention can be contacted with a target cell (e.g. a cell that is not the same as the cell of origin for the exosome) such that the exosome is taken up by the target cell, e.g. endocytosed. Once inside the cell, the contents of the vesicle may be released into the cytoplasm where the molecules contained within the vesicle may act as signaling molecules in one or more signaling pathways thereby inhibiting or enhancing gene expression. The signaling molecules may act at the level of transcription or translation for example. In some instances, where the vesicles contain RNA, the RNA can be transcribed by the target cell. In some instances where the RNA is a miRNA the miRNA can inhibit gene expression.

Methods of Isolating Exosomes

Exosomes may be isolated from any suitable cell that contains exosomes. See e.g., U.S. Pat. No. 10,240,127, which is incorporated herein by reference. Described infra are several exemplary cell and cell types that may be used to implement this method. The method may involve seeding the cell at an appropriate density in a tissue culture vessel and then incubating the cells in a suitable media or buffer for a suitable period of time. In some embodiments the cells may be permitted to attach to the culture vessel before the exosomes are isolated. In other embodiments the cells may be kept in suspension while the exosomes are isolated. The cells may be permitted to replicate in culture before the exosomes are isolated. Alternatively, the exosomes may be isolated from the cells that have not replicated, or replicated minimally (e.g. less than 1 doubling).

To initiate the method the cells are seeded in a tissue culture method at a suitable cell density. The cell density (cells per unit area) may range from about 5 k/cm², about 10 k/cm², about 15 k/cm², about 20 k/cm², about 25 k/cm², about 30 k/cm², about 35 k/cm², about 40 k/cm², about 45 k/cm², about 50 k/cm², about 55 k/cm², about 60 k/cm², about 70 k/cm², about 75 k/cm². In some embodiments the cell density (cells per unit area) may range from about 1 k/cm²-100 k/cm², 10 k/cm²-90 k/cm², 20 k/cm²-80 k/cm², 30 k/cm²-70 k/cm², 40 k/cm²-60 k/cm2. In one embodiment the cells are seeded at a density (cells per unit area) of 40 k/cm².

The cells may be seeded in any isotonic solution. In one embodiment a suitable solution may include a suitable buffer. Examples of suitable buffers may include phosphate buffered saline (PBS), HEPES and the like. In other embodiments the cells may be seeded in any suitable cell culture media, many of which are commercially available. Exemplary media include DMEM, RPMI, MEM, Media 199, HAMS and the like. In one embodiment the media is EGM-MV2. The media may be supplemented with one or more of the following: growth factors, cytokines, hormones, serum, such as fetal calf serum, serum substitutes such as knock out replacement serum or B27, antibiotics, vitamins and/or small molecule drugs. In one embodiment the media is supplemented with a TGF β inhibitor, e.g. SB43154).

The method may be practiced by placing the cells in a suitable environment, such as a cell incubator heated to about 37 degrees C. In some embodiments the cells may be incubated at room temperature. The incubator may be humidified and have an atmosphere that is about 5% CO₂ and about 1% O₂. In some embodiments the CO₂ concentration may range from about 1-20%, 2-10%, 3-5%. In some embodiments the O₂ concentration may range from about 1-20%, 2-10%, 3-5%.

The method may be practiced by incubating the cells in the media or buffer for about 1-72 hours, 1-48 hours, 2-24 hours, 3-18 hours, 4-16 hours, 5-10 hours. In some embodiments the cells are incubated for about 16 hours.

Incubation of the cells as described above allows for the exocytosis of the exosomes by the cells into the isotonic solution. After incubation of the cells in the isotonic solution as described above, the isotonic solution may be harvested for exosomes. Exosomes are purified using methods described (e.g., Example 1).

Progenitor Cells

In certain embodiments of the invention progenitor cells serve as the source of the exosomes described infra. The progenitor cell may be from any animal or plant. For example the exosome may be from a mammal, such as a human, a non-human primate, a horse, a cow, a sheep, a goat, a pig, a cat, a dog, a rabbit, a guinea pig, a rodent such as a mouse or a rat. Typically a progenitor cell will not have an essentially unlimited replicative capacity as typically found in embryonic stem cells, but will nonetheless have, a result of their longer telomeres, a greater replicative capacity compared to adult primary cells or tissues (e.g. primary cells) or adult stem cells.

The progenitor cell may be derived from a pluripotent stein cell, such as an embryonic stem cell or an induced pluripotent stem cell. The progenitor cell may be a clonal cell or an oligoclonal cell. An oligoclonal cell would include a population of cells similar cells, e.g. phenotypically or genetically. The progenitor cell may be a clonal human embryonic progenitor cell. The progenitor cell may be a clonal human embryonic endothelial progenitor cell. In a preferred embodiment, the progenitor cell line is 30-MV2-14, 30-MV2-4, E69, or RPI-MV2-8.

Where the progenitor cells are clonal cells obtained from pluripotent stem cells they will provide an almost unlimited source of the same exosomes. This is due to two factors: the genetic identity of the original cellular source material and the enhanced telomere lengths found in early progenitors which provide for enhanced replicative capacity relative to adult tissue or cells or adult stem cells. Moreover, unlike adult stem cells which are typically available in very small numbers and are difficult to expand in culture, the clonal embryonic progenitors described infra are available in large numbers and are relatively easy to expand in culture.

Uses of Exosomes

The exosomes described herein may be used in therapeutic, research and diagnostic applications. For example the exosomes described infra may be added to a cell culture to enhance one or more phenotypic traits of the cells. The exosomes of the invention may be added to a cell culture to inhibit one or more phenotypic traits of the cells. The exosomes of the invention may be added to a cell culture to provide a new phenotypic trait of the cells.

The exosomes of the invention may be added to a culture of endothelial cells to enhance the ability of the cells to form vascular tube like structures. The exosomes of the invention may be added to any cell having the ability to form vascular tube like structures to enhance the cells ability to form tube like structures.

In some embodiments the exosomes of the invention are contacted with a cell thereby providing at least one new phenotypic trait to the cell. For example, the exosomes of the invention may confer the ability to form vascular tube like structures to cell lacking the ability to form vascular tube like structures before it was contacted with the exosomes of the invention.

In certain embodiments the exosomes of the invention may be added to a culture of perivascular cells to enhance the ability of the perivascular cells to form vascular tube like structures.

In some embodiments the invention provides a method of increasing the length of a vascular tube like structure formed by a cell such as an endothelial relative to an endothelial cell that has not been treated with the exosomes of the invention comprising contacting the endothelial cell with an exosome isolated from a progenitor cell such as a human clonal progenitor cell, e.g., 30-MV2-14, 30-MV2-4, E69, or RPI-MV2-8 cells. In some embodiments the invention provides a method of increasing the length of a vascular tube like structure formed by a cell such as a perivascular cell relative to a perivascular cell that has not been treated with the exosomes of the invention comprising contacting the perivascular cell with an exosome isolated from a progenitor cell such as a human clonal progenitor cell, e.g., 30-MV2-14, 30-MV2-4, E69 or RPI-MV2-8 cells. In some embodiments the invention provides a method of increasing the branching of a vascular tube like structure formed by an endothelial cell relative to an endothelial cell that has not been treated with the exosomes of the invention comprising contacting the endothelial cell with an exosome isolated from a progenitor cell such as a human clonal progenitor cell, e.g., 30-MV2-14, 30-MV2-4, E69 or RPI-MV2-8 cells. In some embodiments the invention provides a method of increasing the branching of a vascular tube like structure formed by a perivascular cell relative to a perivascular cell that has not been treated with the exosomes of the invention comprising contacting the perivascular cell with an exosome isolated from a progenitor cell such as a human clonal progenitor cell, e.g., 30-MV2-14, 30-MV2-4, E69 or RPI-MV2-8 cells. In still other embodiments the invention provides a method of increasing the number of loops in the vascular tube like structures formed by an endothelial cell relative to an endothelial cell that has not been treated with the exosomes of the invention comprising contacting the endothelial cell with an exosome isolated from a progenitor cell such as a human clonal progenitor cell, e.g., 30-MV2-14, 30 MV2-4, E69 or RPI-MV2-8 cells. In yet other embodiments the invention. provides a method of increasing the number of loops in the vascular tube like structures formed by a perivascular cell relative to a perivascular cell that has not been treated with the exosomes of the invention comprising contacting the perivascular cell with an exosome isolated from a progenitor cell such as a human clonal progenitor cell, e.g., 30-MV2-14, 30-MV2-4, E69 or RPI-MV2-8 cells.

The exosomes of the invention may be administered therapeutically to a subject in need of treatment. For example the exosomes of the invention may be administered to a subject in need of treatment for any disease requiring the enhanced ability to form vascular tube like structures. The exosomes of the invention may be used to treat a subject suffering from cardiovascular disease, heart failure, infarction, chronic wounds, ulcer, clogged vessels or arteries, damaged vessels, stenotic vessels, arteriosclerosis, angina, peripheral vascular disease, Alzheimer's disease, ischemia, diabetes, cancer, cell replacement transplant or therapy, tissue and cell regenerative therapy and Parkinson's disease. The exosomes may be used as depot to deliver therapeutic molecules such as small molecules, nucleic acids, proteins and peptides.

The exosomes of the invention may be directly administered to a subject in need of treatment or an in vitro cell culture. Alternatively the exosomes can be provided enclosed within a matrix or scaffold. Suitable matrices or scaffolds may include a matrix or scaffold comprised of one or more extracellular matrix proteins, e.g. laminin, fibronectin and the like. Other suitable matrices or scaffolds include Matrigel® which is a murine sarcoma extract. The matrix or scaffold may be a hydrogel. The hydrogel may be comprised of hylauronate and gelatin (see U.S. Pat. Nos. 8,324,184; 7,928,069). In one embodiment the exosomes of the invention may be delivered in HyStem (Lineage Cell Therapeutics, Inc., Alameda Calif.).

Using the methods described infra along with routine chromatographic techniques known in the art the exosomes of the invention may be used to isolate one or more nucleic acids, proteins or peptides expressed by a progenitor cell serving as the source of the exosome. Once isolated, the proteins or peptides isolated from the exosomes of the invention can be used to make antibodies to the isolated proteins or peptides (See Harlow et al. Antibodies: A Lab Manual 2.sup.nd Edition; Cold Spring Harbor Press 2013).

The exosomes of the invention may be used in drug screening assays. For example where the exosomes described infra enhance vascular tube formation in vitro, the exosomes can be used to screen for drugs that enhance or inhibit this capability. A cell culture comprising cells having the ability to form vascular tube like structures may be contacted with the exosomes of the invention and a drug candidate may be applied to the same cell culture either before, after or simultaneously with the exosomes to determine the effect of the drug the ability of the exosomes to enhance vascular tube formation in the cell culture. The effects can be compared to untreated cells and cells treated only with the exosomes of the invention.

The exosomes of the present invention may be used to reduce the number of senescent cells in a population. The exosomes of the present invention may be used to reduce the amount of senescence associated secretory phenotype (SASP) proteins produced by a cell population.

Pharmaceutical Compositions

Modes of administration for a therapeutic (either alone or in combination with other pharmaceuticals) can be, but are not limited to, sublingual, injectable (including short-acting, depot, implant and pellet forms injected subcutaneously or intramuscularly), or by use of vaginal creams, suppositories, vaginal rings, rectal suppositories, intrauterine devices, and transdermal forms such as patches and creams.

Specific modes of administration will depend on the indication. The selection of the specific route of administration and the dose regimen is to be adjusted or titrated by the clinician according to methods known to the clinician in order to obtain the optimal clinical response. The amount of therapeutic to be administered is that amount which is therapeutically effective. The dosage to be administered will depend on the characteristics of the subject being treated, e.g., the particular animal treated, age, weight, health, types of concurrent treatment, if any, and frequency of treatments, and can be easily determined by one of skill in the art (e.g., by the clinician).

Pharmaceutical formulations containing the therapeutic of the present disclosure and a suitable carrier can be solid dosage forms which include, but are not limited to, tablets, capsules, cachets, pellets, pills, powders and granules; topical dosage forms which include, but are not limited to, solutions, powders, fluid emulsions, fluid suspensions, semi-solids, ointments, pastes, creams, gels and jellies, and foams; and parenteral dosage forms which include, but are not limited to, solutions, suspensions, emulsions, and dry powder; comprising an effective amount of a polymer or copolymer of the present disclosure. It is also known in the art that the active ingredients can be contained in such formulations with pharmaceutically acceptable diluents, fillers, disintegrants, binders, lubricants, surfactants, hydrophobic vehicles, water soluble vehicles, emulsifiers, buffers, humectants, moisturizers, solubilizers, preservatives and the like. The means and methods for administration are known in the art and an artisan can refer to various pharmacologic references for guidance. For example, Modern Pharmaceutics, Banker & Rhodes, Marcel Dekker, Inc. (1979); and Goodman & Gilman's The Pharmaceutical Basis of Therapeutics, 6th Edition, MacMillan Publishing Co., New York (1980) can be consulted.

The compositions of the present disclosure can be formulated for parenteral administration by injection, e.g., by bolus injection or continuous infusion. The compositions can be administered by continuous infusion subcutaneously over a period of about 15 minutes to about 24 hours. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents.

For oral administration, the compositions can be formulated readily by combining the therapeutic with pharmaceutically acceptable carriers well known in the art. Such carriers enable the therapeutic of the invention to be formulated as tablets, pills, dragees, capsules, liquids, gels, syrups, slurries, suspensions and the like, for oral ingestion by a patient to be treated. Pharmaceutical preparations for oral use can be obtained by adding a solid excipient, optionally grinding the resulting mixture, and processing the mixture of granules, after adding suitable auxiliaries, if desired, to obtain tablets or dragee cores. Suitable excipients include, but are not limited to, fillers such as sugars, including, but not limited to, lactose, sucrose, mannitol, and sorbitol; cellulose preparations such as, but not limited to, maize starch, wheat starch, rice starch, potato starch, gelatin, gum tragacanth, methyl cellulose, hydroxypropylmethyl-cellulose, sodium carboxymethylcellulose, and polyvinyl pyrrolidone (PVP). If desired, disintegrating agents can be added, such as, but not limited to, the cross-linked polyvinyl pyrrolidone, agar, or alginic acid or a salt thereof such as sodium alginate.

Dragee cores can be provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active therapeutic doses.

Pharmaceutical preparations which can be used orally include, but are not limited to, push-fit capsules made of gelatin, as well as soft, sealed capsules made of gelatin and a plasticizer, such as glycerol or sorbitol. The push-fit capsules can contain the active ingredients in admixture with filler such as, e.g., lactose, binders such as, e.g., starches, and/or lubricants such as, e.g., talc or magnesium stearate and, optionally, stabilizers. In soft capsules, the active therapeutic can be dissolved or suspended in suitable liquids, such as fatty oils, liquid paraffin, or liquid polyethylene glycols. In addition, stabilizers can be added. All formulations for oral administration should be in dosages suitable for such administration.

For buccal administration, the pharmaceutical compositions can take the form of, e.g., tablets or lozenges formulated in a conventional manner.

For administration by inhalation, the therapeutic for use according to the present disclosure is conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of, e.g., gelatin for use in an inhaler or insufflator can be formulated containing a powder mix of the therapeutic and a suitable powder base such as lactose or starch.

The compositions of the present disclosure can also be formulated in rectal compositions such as suppositories or retention enemas, e.g., containing conventional suppository bases such as cocoa butter or other glycerides.

In addition to the formulations described previously, the therapeutic of the present disclosure can also be formulated as a depot preparation. Such long acting formulations can be administered by implantation (for example subcutaneously or intramuscularly) or by intramuscular injection.

Depot injections can be administered at about 1 to about 6 months or longer intervals. Thus, for example, the compositions can be formulated with suitable polymeric or hydrophobic materials (for example as an emulsion in an acceptable oil) or ion exchange resins, or as sparingly soluble derivatives, for example, as a sparingly soluble salt.

In transdermal administration, the compositions of the present disclosure, for example, can be applied to a plaster, or can be applied by transdermal, therapeutic systems that are consequently supplied to the organism.

Pharmaceutical compositions can include suitable solid or gel phase carriers or excipients. Examples of such carriers or excipients include but are not limited to calcium carbonate, calcium phosphate, various sugars, starches, cellulose derivatives, gelatin, and polymers such as, e.g., polyethylene glycols.

The compositions of the present disclosure can also be administered in combination with other active ingredients, such as, for example, adjuvants, protease inhibitors, or other compatible drugs or compounds where such combination is seen to be desirable or advantageous in achieving the desired effects of the methods described herein.

In some embodiments, the disintegrant component comprises one or more of croscarmellose sodium, carmellose calcium, crospovidone, alginic acid, sodium alginate, potassium alginate, calcium alginate, an ion exchange resin, an effervescent system based on food acids and an alkaline carbonate component, clay, talc, starch, pregelatinized starch, sodium starch glycolate, cellulose floc, carboxymethylcellulose, hydroxypropylcellulose, calcium silicate, a metal carbonate, sodium bicarbonate, calcium citrate, or calcium phosphate.

In some embodiments, the diluent component may include one or more of mannitol, lactose, sucrose, maltodextrin, sorbitol, xylitol, powdered cellulose, microcrystalline cellulose, carboxymethylcellulose, carboxyethylcellulose, methylcellulose, ethylcellulose, hydroxyethyl cellulose, methylhydroxyethyl cellulose, starch, sodium starch glycolate, pregelatinized starch, a calcium phosphate, a metal carbonate, a metal oxide, or a metal aluminosilicate.

In some embodiments, the optional lubricant component, when present, comprises one or more of stearic acid, metallic stearate, sodium stearylfumarate, fatty acid, fatty alcohol, fatty acid ester, glycerylbehenate, mineral oil, vegetable oil, paraffin, leucine, silica, silicic acid, talc, propylene glycol fatty acid ester, polyethoxylated castor oil, polyethylene glycol, polypropylene glycol, polyalkylene glycol, polyoxyethylene-glycerol fatty ester, polyoxyethylene fatty alcohol ether, polyethoxylated sterol, polyethoxylated castor oil, polyethoxylated vegetable oil, or sodium chloride.

Kits

In some embodiments the invention provides a kit comprising exosomes isolated from a progenitor cell, such as a human clonal progenitor cell. The progenitor cell may be an endothelial progenitor cell, such as human clonal embryonic progenitor cell, e.g. 30-MV2-14, 30-MV2-4, E69 or RPI-MV2-8. The exosomes may be provided in one or more containers. The exosomes may be provided in a suitable buffer, e.g. PBS or a suitable media, such as a commercially available cell culture media, e.g. DMEM. The kit may further contain a cell having the ability to form vascular tube like structures. The cell may be an endothelial cell, e.g. HUVEC and/or a perivascular cell. The cells may be provided in a suitable media, e.g. DMEM or the like or alternatively the cells may be provided in a buffer such as PBS. In some embodiments the cells may be provided frozen in a suitable freezing media such as a commercially available media supplemented with DMSO. The kit may optionally include instructions as to how to reconstitute the exosomes, culture the cells and/or contact the cells with exosomes so as to enhance vascular tube like formation.

In other embodiments the invention provides a kit comprising a human clonal embryonic progenitor cell, such as 30-MV2-14, 30-MV2-4, E69 or RPI-MV2-8. The cell may be provided in at least one container in suitable media or buffer. The kit may include buffers and/or media for isolating exosomes from the cells. The kit may contain one or more vessels, e.g. a multi-well plate for culturing the cells. The kit may further contain a cell line capable of forming vascular tube like structures such as endothelial cells. Suitable cells include endothelial cells such as HUVEC and/or a perivascular cell. Any or all of the cells may be provided frozen in a suitable media, e.g. freezing media such as a commercially available media supplemented with DMSO. The kit may optionally include instructions as to how to culture the cells and/or contact the endothelial cells with exosomes isolated from the progenitor cells so as to enhance or induce vascular tube like formation.

### Example 1: Purification of Exosomes from Clonal Progenitor Cell Lines

PureStem Endothelial Progenitor Cells (available from AgeX Therapeutics, Inc.; West et al. (2008) Regen Med 3:287) were maintained in endothelial growth medium (EGM-MV2, PromoCell, GmbH, Germany) on Gelatin-coated plates. The medium was changed every 2-3 days and cells were passaged at 80-90% confluence with TrypLE Express medium. Cells used for exosome collection were between passages 10 and 13 for EV collection. After cells reached ˜80% confluence, cells were washed two times with PBS. Medium was changed with conditioned medium containing endothelial basal medium (EBM) supplement with VEGF, IGF and FGF, and cultures were incubated for 72 hours at 5% oxygen.

Conditioned media were centrifuged at 300 g for 5 min followed by 1000 g for 10 min at room temperature and filtered through 0.2 um to remove cells and cellular debris. Conditioned medium was then subjected to ultrafiltration in Tangential Flow Filtration (TFF) system using a 100 kDa cutoff TFF cartridge (PALL Laboratory, New York). A feed flow rate of 40 mL/min with transmembrane pressure <2 psi was applied. The conditioned medium was concentrated 10-fold and centrifuged at 10,000 g for 10 min. Size exclusion chromatography (SEC) using qEV100 columns (Izon Science, Cambridge, Mass.) was performed for further purification of exosomes. Briefly, after rinsing the qEV columns with PBS, 100 ml of TFF-concentrated exosomes were eluted with 6 fractions (F1-F6, total 150 ml). A total of F1-F6 fractions were pooled and further concentrated. Amicon Ultrafilter-70 Centrifugal Filters (100 KDa MWCO, Millipore, Mass.) to concentrate exosomes. Purified exosomes were aliquoted at 100 uL each and stored at −80 C.

The size distribution and particle concentration of exosomes were measured using the Tunable Resistive Pulse Sensing (TRPS) qNano platform (iZON® Science, UK). The instrument was set up and calibrated as per manufacturer recommendations. A polyurethane nanopore (NP150, Izon Science) was used and axially stretched to 47 mm, as measured on the qNano unit. Data processing and analysis were carried out on Izon Control Suite software v3.3 (Izon Science).

The purified exosomes were resuspended in 100 uL of PBS, lysed in RIPA buffer, and then measured for protein quantity by a bicinchoninic acid (BCA) assay using the Micro BCA Protein Assay Kit (Thermo) according to the manufacturer's instructions. Exosome protein content was determined by calibration against a standard curve, which was prepared by plotting the absorbance at 562 nm versus BSA standard concentration.

### Example 2: Migration Assay

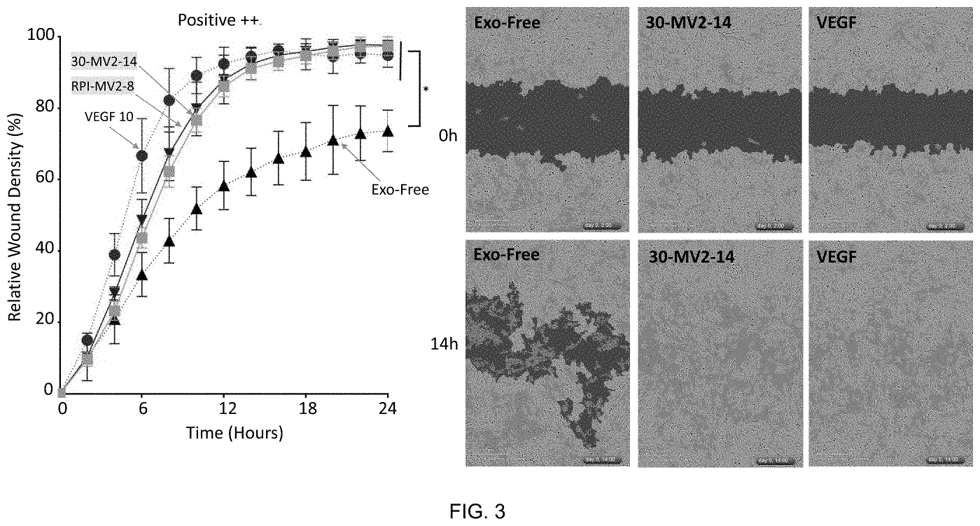

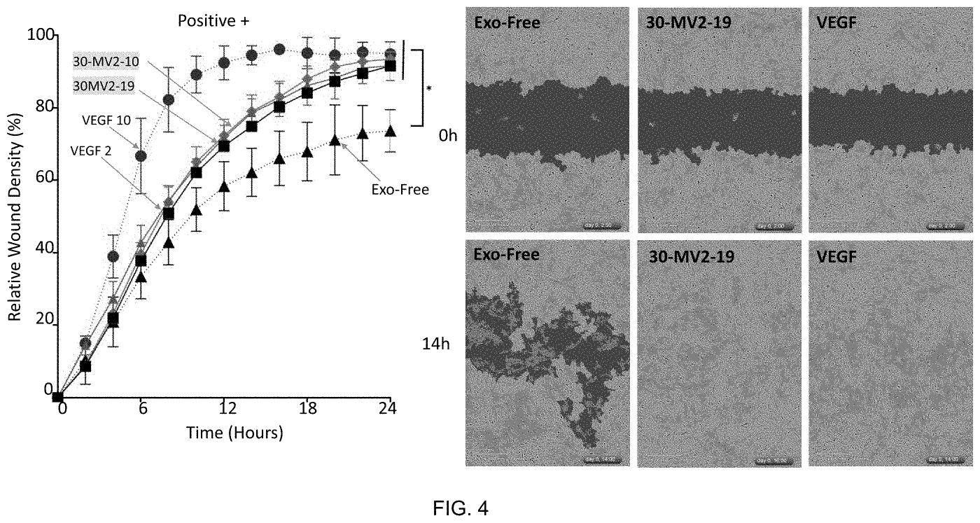

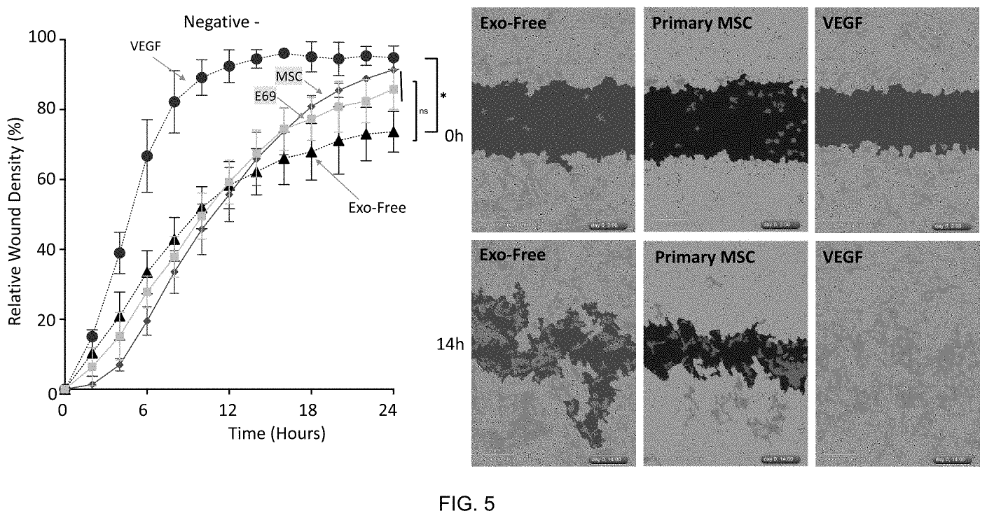

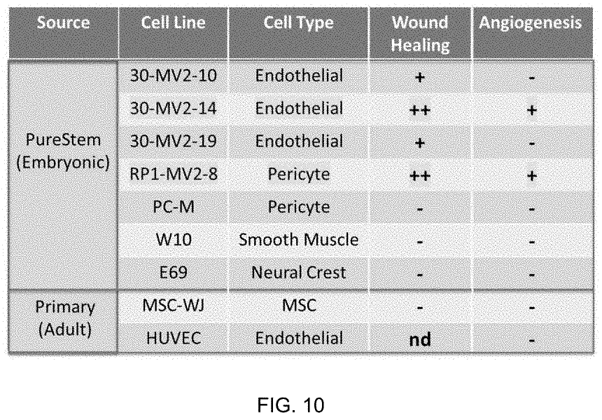

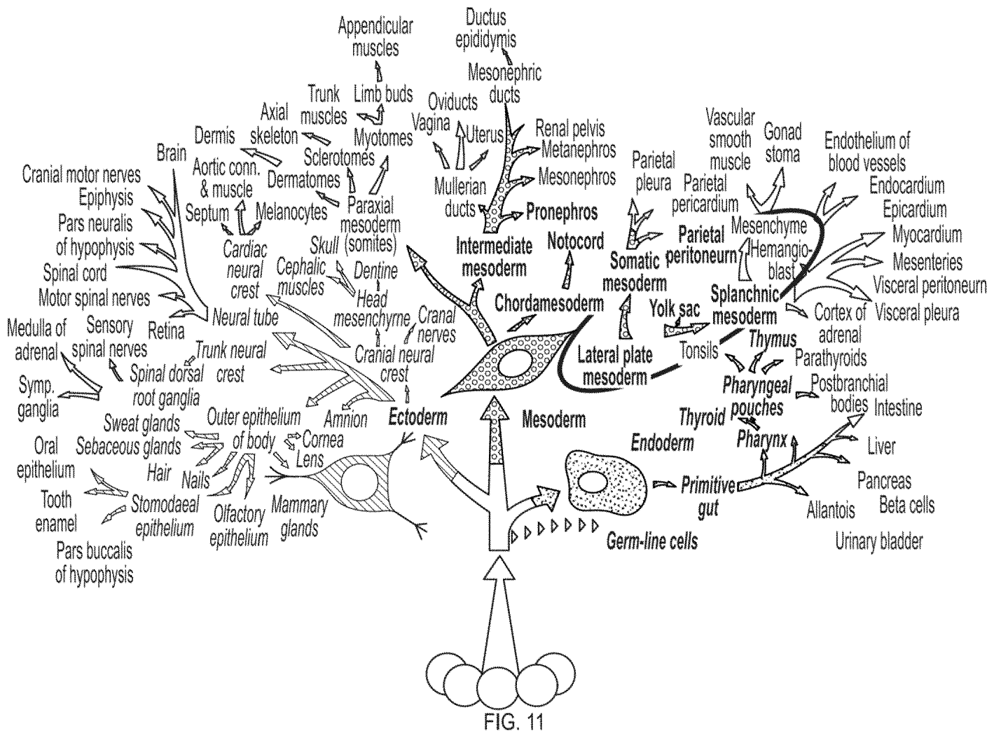

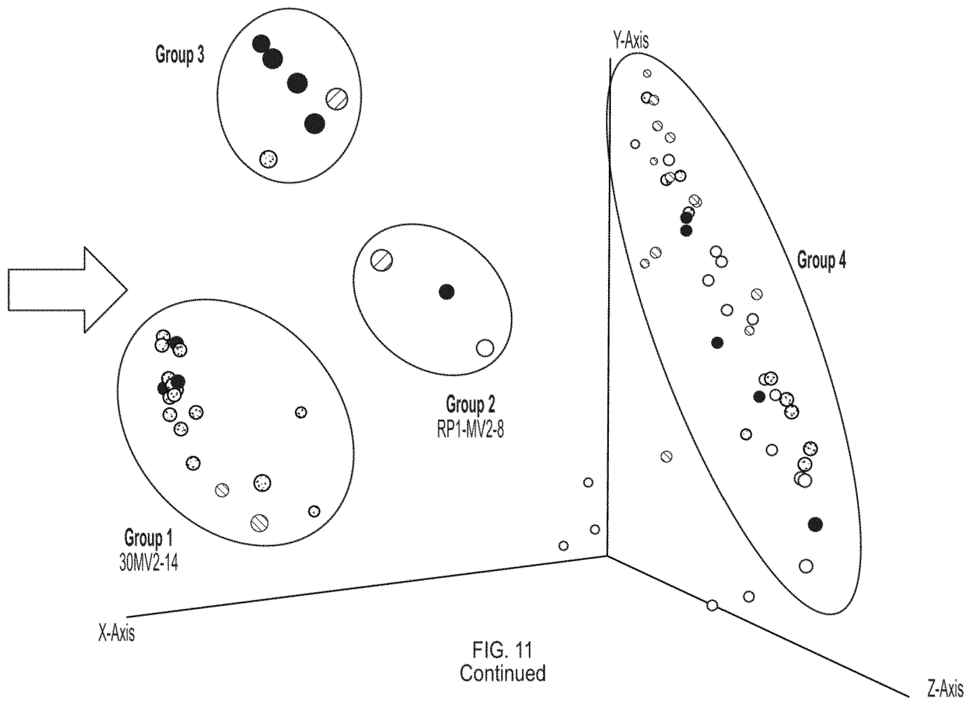

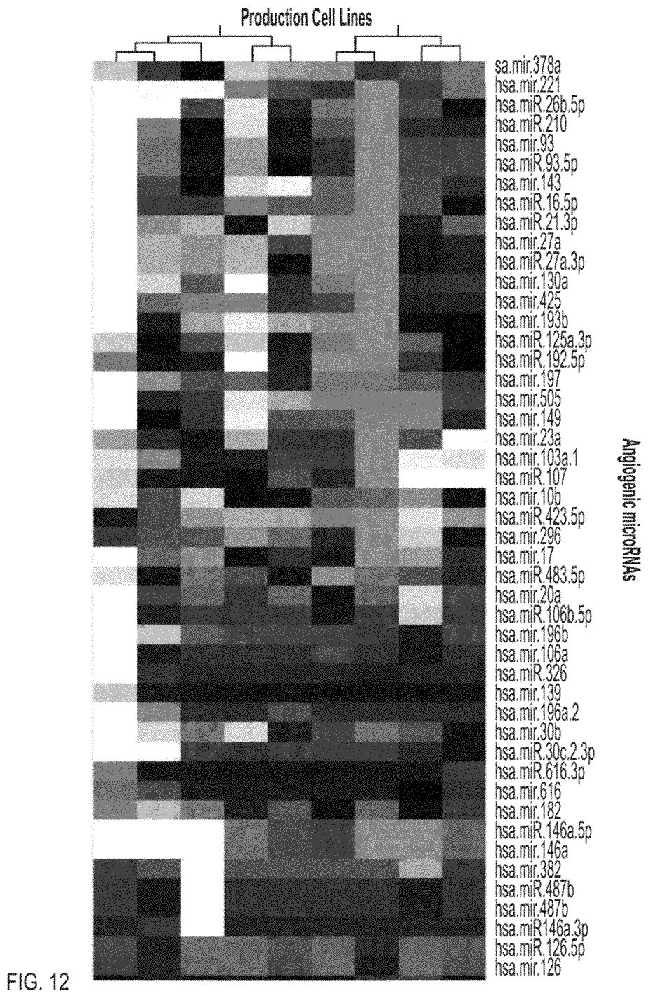

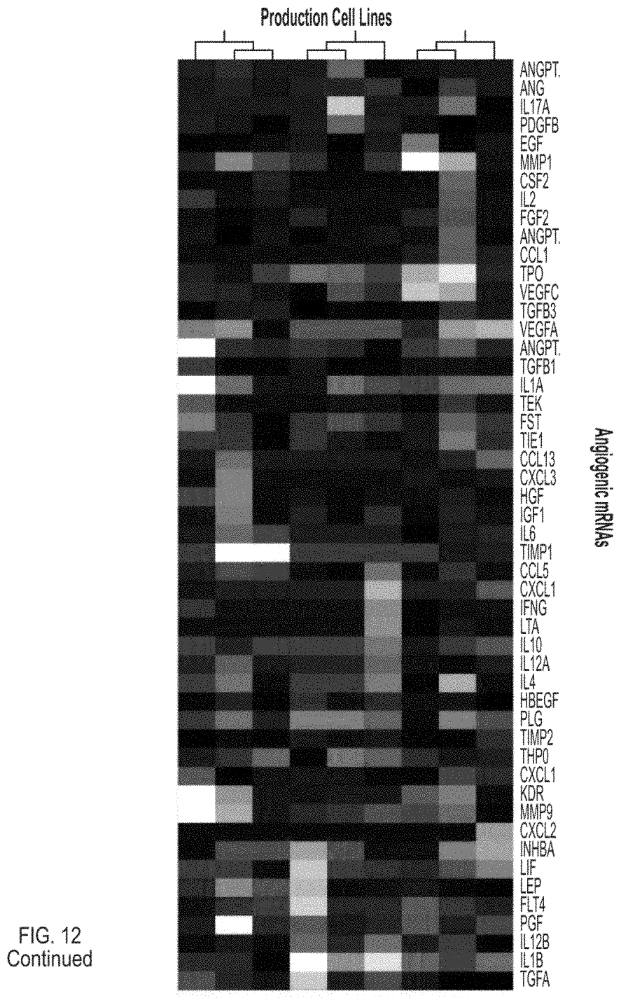

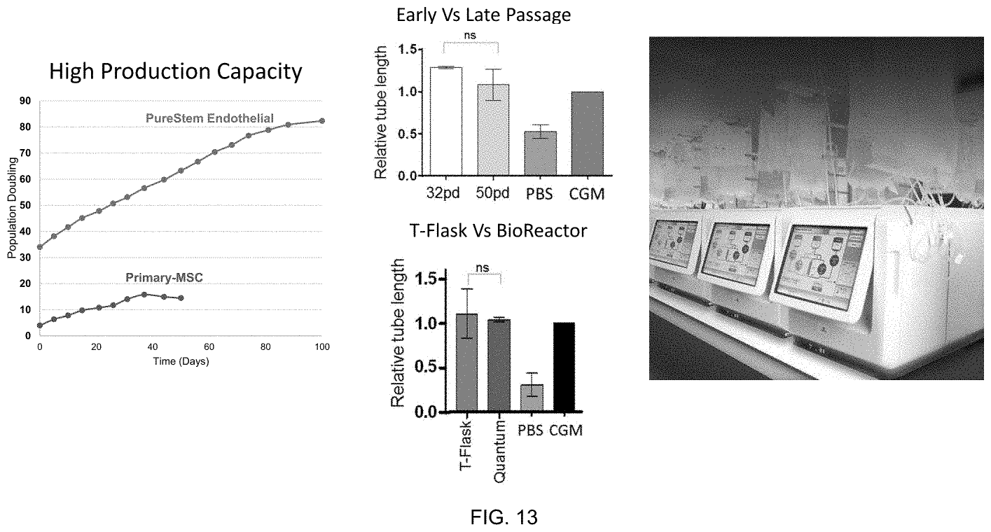

Cell migration was assessed using a scratch wound healing assay format. HUVEC (1E4 cells per well) were plated onto 0.1% gelatin coated 96-well plates, and the following day a scratch was made on confluent monolayers using a 96-pin WoundMaker (Essen BioScience, Ann Arbor, Mich.). Exosomes (2E7, 4E7 and 1.2E8 particles per well) and growth factor (i.e. 4 ng/ml VEGF as a positive control) were treated with exosome-depleted EGM-MV2. Wound images were automatically acquired by the IncuCyte software system every 2 hours for 24 hours. Wound closure and cell confluence were calculated using the IncuCyte 96-Well Cell Migration Software Application Module. Migration data were analyzed as the Percent of Relative Wound Density (% RWD). RWD is a representation of the spatial cell density in the wound area relative to the spatial cell density outside of the wound area at every time point (time-curve). See FIGS. 3-5 and 10.

### Example 3: Angiogenesis Assay

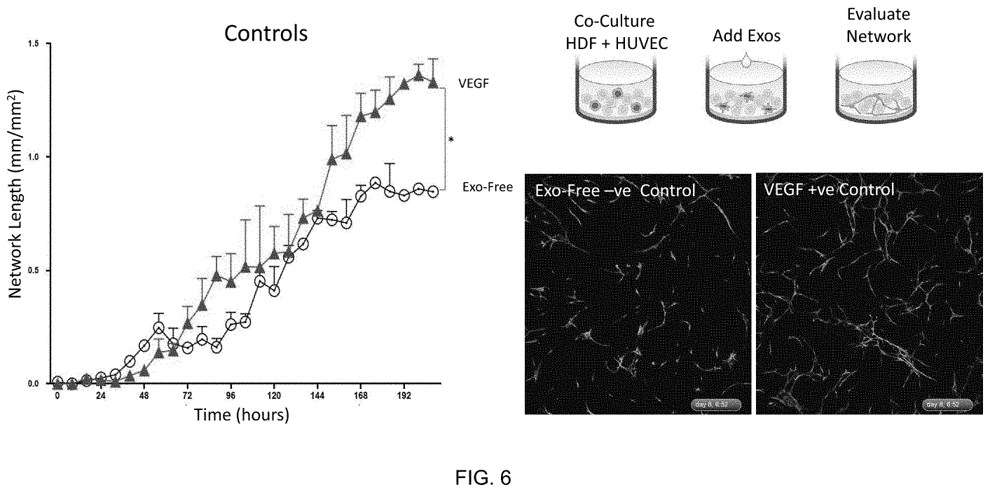

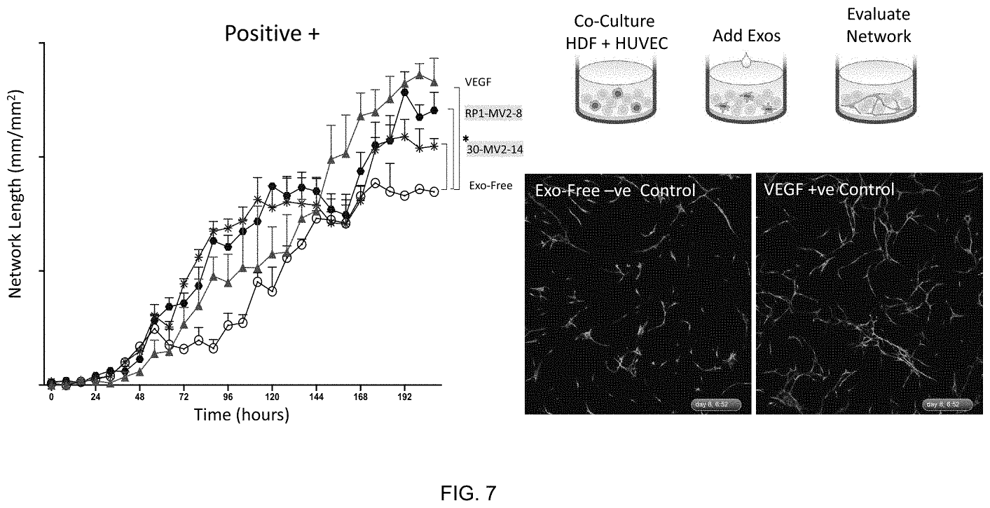

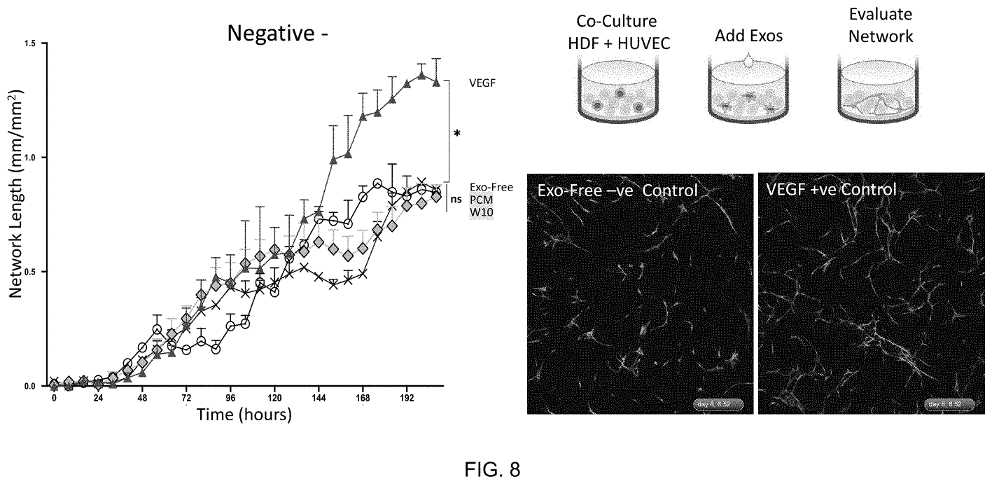

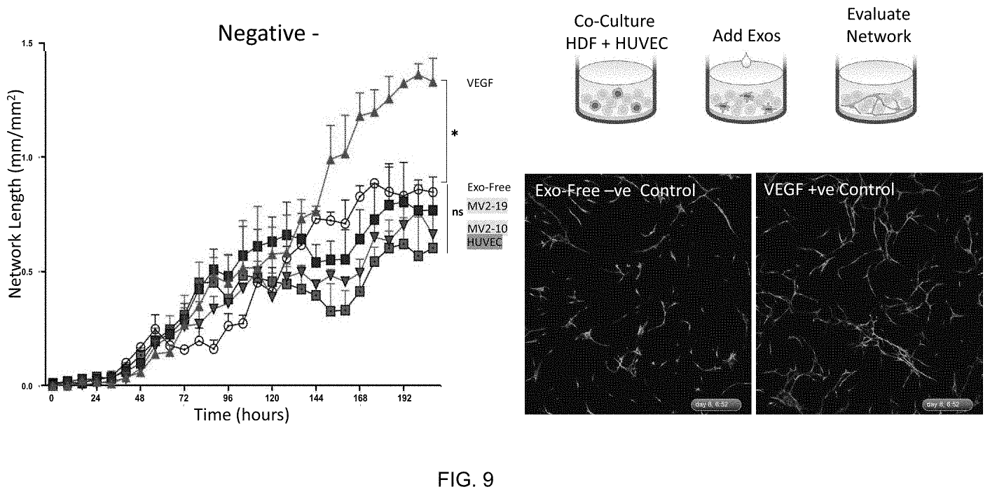

The CellPlayer Angiogenesis PrimeKit (Essen BioScience) was performed according to the manufacture's protocol. On day 0, normal human dermal fibroblasts (NHDFs) were plated into a 96-well plate and then incubated at room temperature in a tissue culture hood for 1 hour to allow them to adhere to the plate. The HUVEC-CytoLight Green were then plated onto the NHDF feeder layers and incubated at room temperature for 1 hour prior to placing in the IncuCyte (Essen BioScience) for imaging. The next day, treatment initiated with a media change including exosomes (4E7 particles per well) and growth factor (i.e. 4 ng/ml VEGF as a positive control) in exosome-depleted EGM-MV2. Cultures were then fed every 3 days at which time complete media changes occurred with fresh growth factor and exosome addition. Following seeding, co-cultures were placed in an IncuCyte live imaging system, and images were automatically acquired in both phase and fluorescence every 6 hours for 10 to 14 days at 10× objective magnification using the tiled field of view mosaic imaging mode. In this mode, six images were acquired per well and merged into a single larger image. Tube formation over the 14 days was quantified using the IncuCyte Angiogenesis Analysis Module. For analyzing angiogenesis, the metric of tube network length (mm/mm²) was used by measuring lengths of all of the networks in the image divided by the image area at every time point. See FIGS. 6-10.

### Example 4: Exosome Loading Example

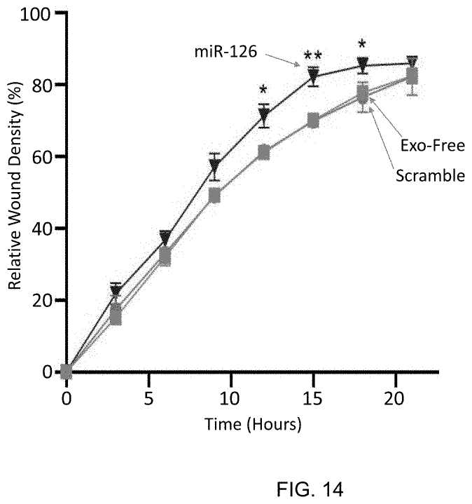

Exosomes were engineered with cargo miRNAs (miR-126-3p) via electroporation performed on a Neon Transfection System (Thermo Fisher Scientific). Isolated exosomes and miRNA were mixed, and the final volume was adjusted to 100 ul using electroporation buffer. The amount of exosomes and miRNA used for electroporation was 1*E\hat{ }8 exosomes and 1 pmol miRNA. The exosome-miRNA mixture was aspirated into 100 ul Neon® Tip with Neon® pipette and electroporated with the following parameters: pulse width of 20 ms, pulse voltage of 1000V and pulse numbers of 10. After delivering the electric pulse, mixture was transferred from Neon® Tip to Amicon® Ultra-0.5 centrifugal filter devices (Millipore; 30,000 MWCO) to remove free miRNAs. Samples were spun at 10,000×g for 15 minutes. Engineered exosomes were recovered into a clean microcentrifuge tube by placing filter device upside down and spin for 2 minutes at 1,000×g. See FIG. 14.

### Example 5: Characterization of Exosomes, Functions, Purity, Proteins, Protein Utilities, miRNA and miRNA Functions

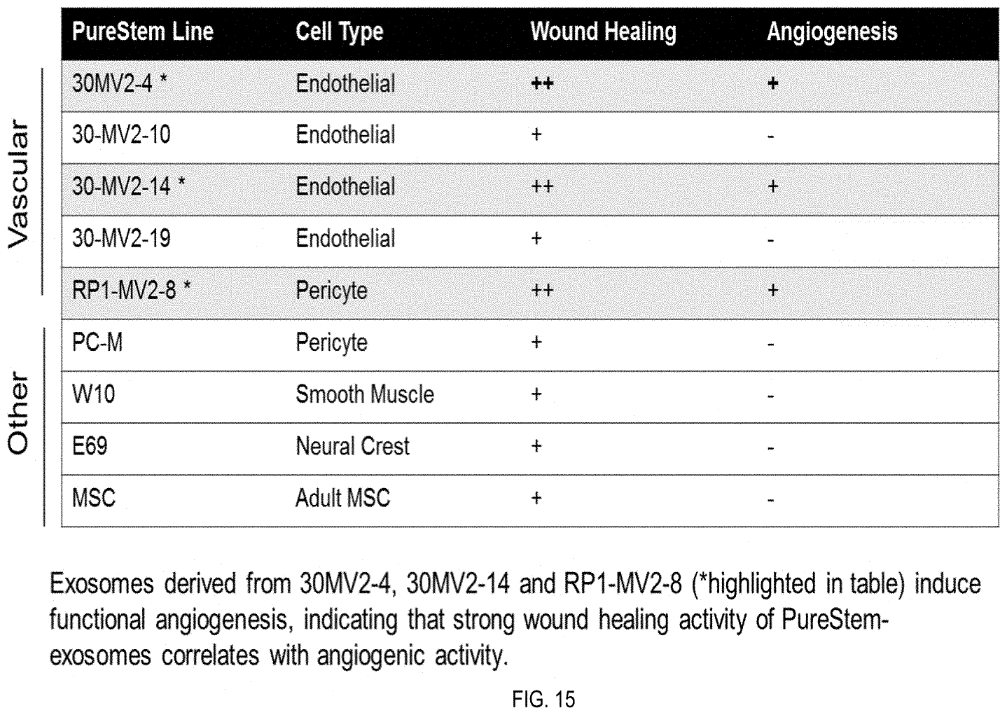

In addition, FIG. 15 provides a summary showing exosomes derived from 30-MV2-4, 30-MV2-14, and RP1-MV2-8 induce functional angiogenesis, indicating that strong wound healing activity of PureStem-exosomes correlates with angiogenic activity.

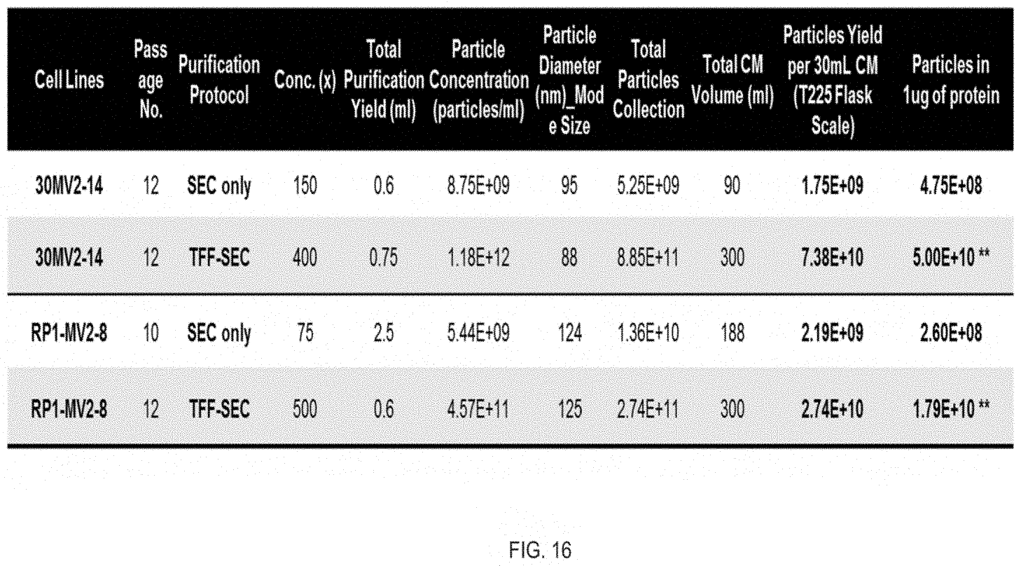

FIG. 16 shows that using the developed protocols applying TFF-SEC exosome isolation method, the presented invention resulted in highly purified exosomes with increasing production yield and purity compared to SEC alone method. The purity was in the range of 1E10-5E10 particles/ug, which meets the Guidelines from ISEV for quality control.

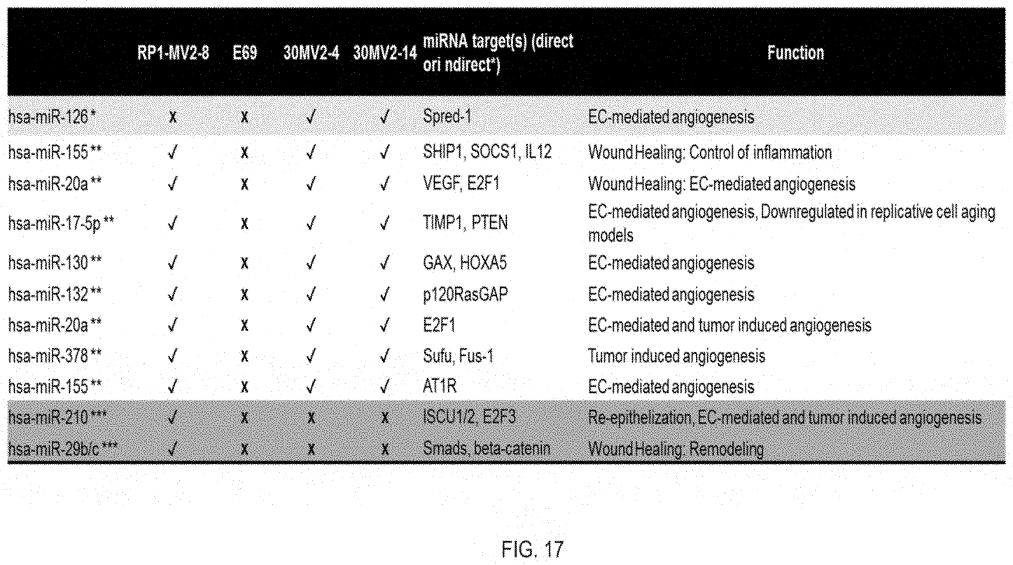

FIG. 17 is a list of miRNAs contained in PureStem-exosomes and their roles. Angiogenic activity is detected in all lines except E69. miRNAs shown are detected in angiogenic exosomes but not in E69 exosomes (no angiogenesis detected). Lines 30-MV2-4 and 30-MV2-14 expressed miRNA*, RP-1-MV2-8, 30-MV2-4, and 30MV2-14 expressed miRNAs**, and only RP-1MV2-8 expressed RNAs***.

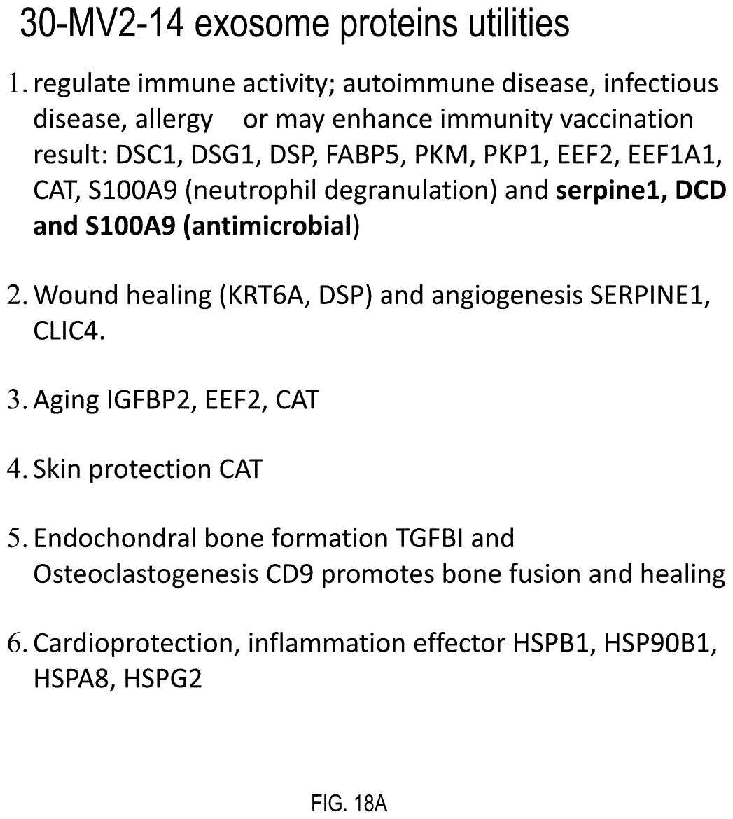

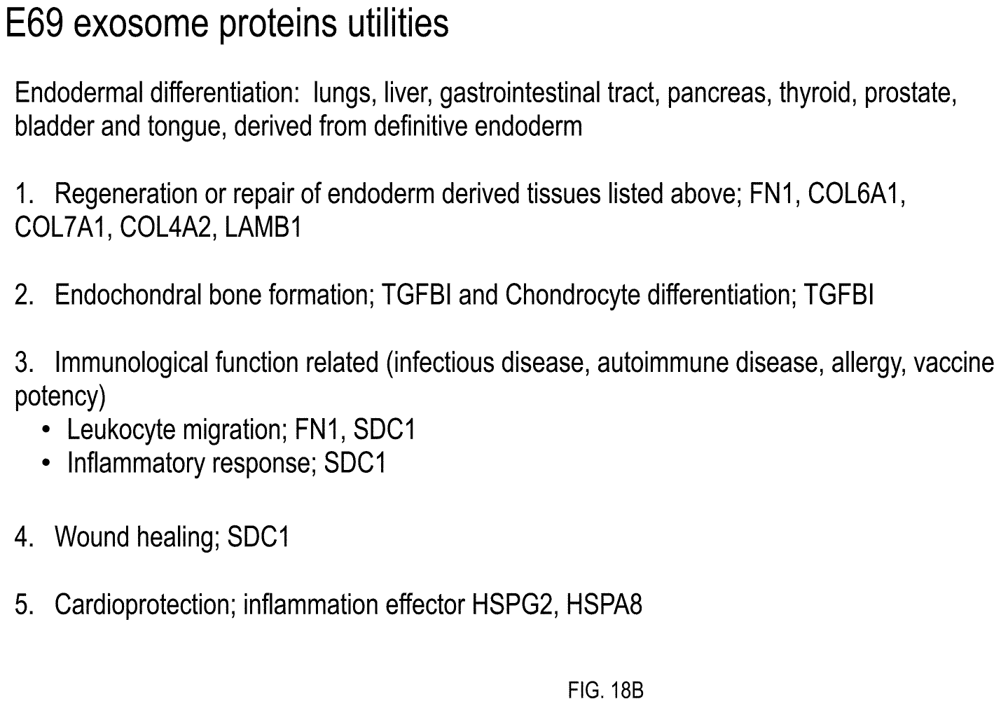

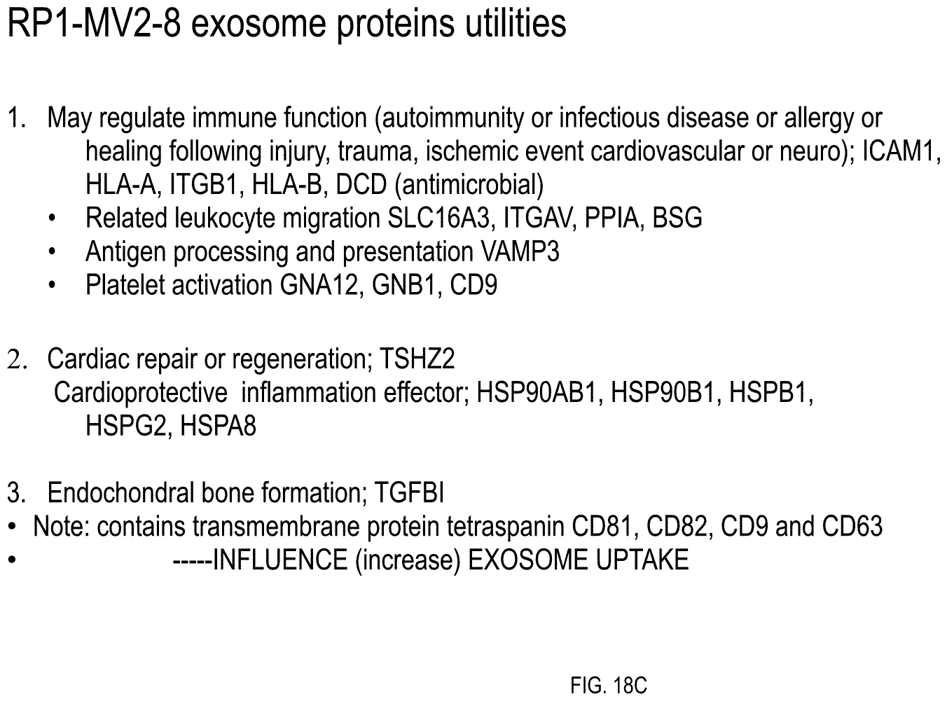

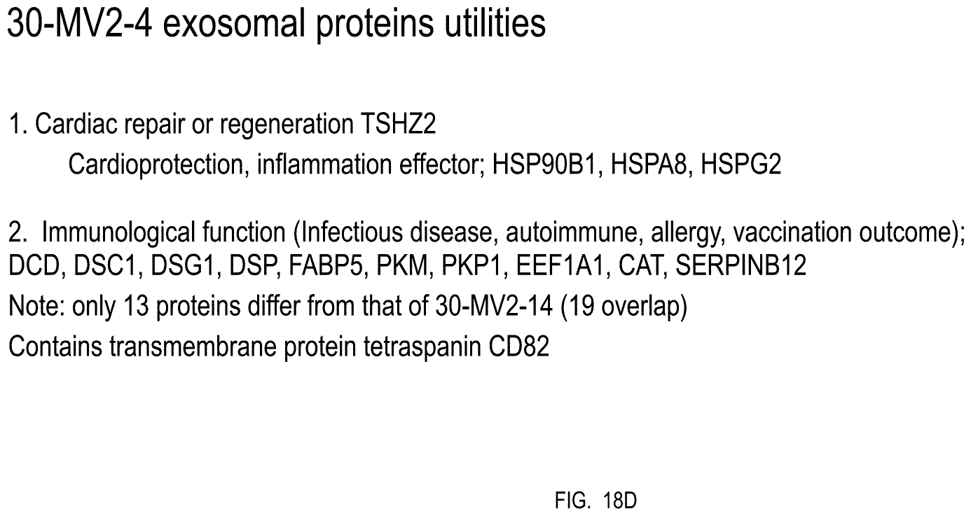

FIG. 18A-D show exosome protein utilities for 30-MV2-14, E69, RP1-MV2-8, and 30-MV2-4.

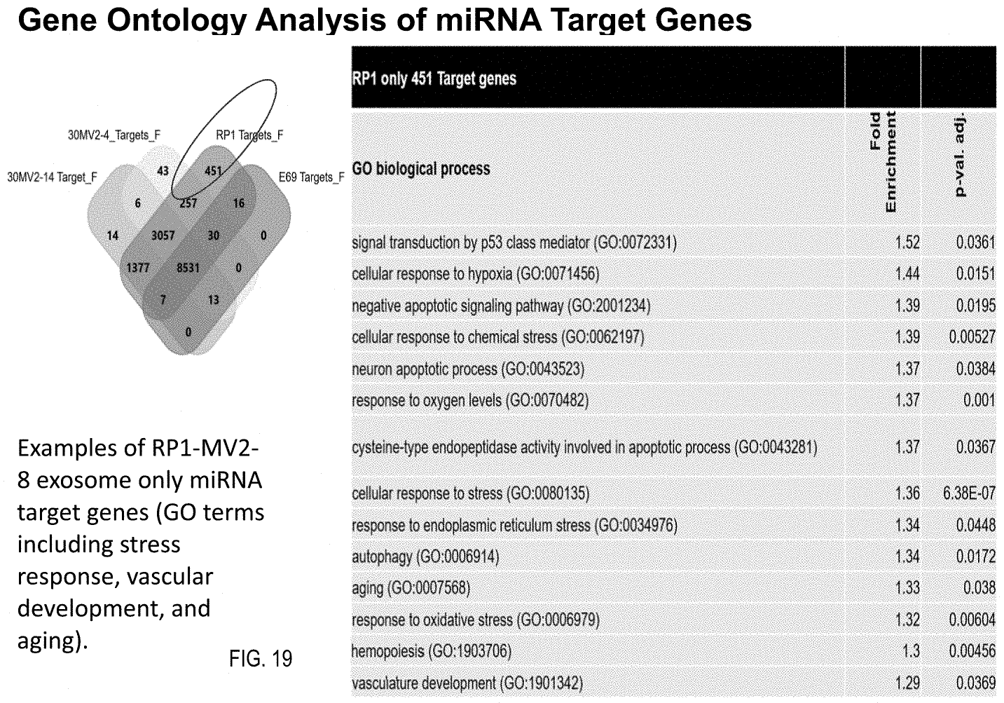

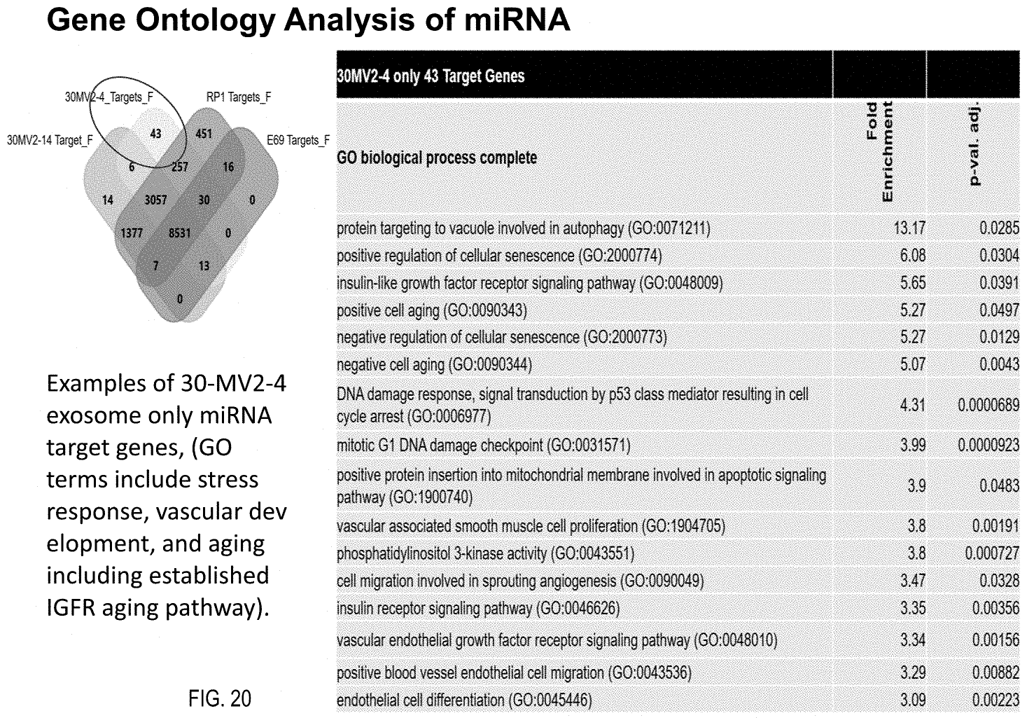

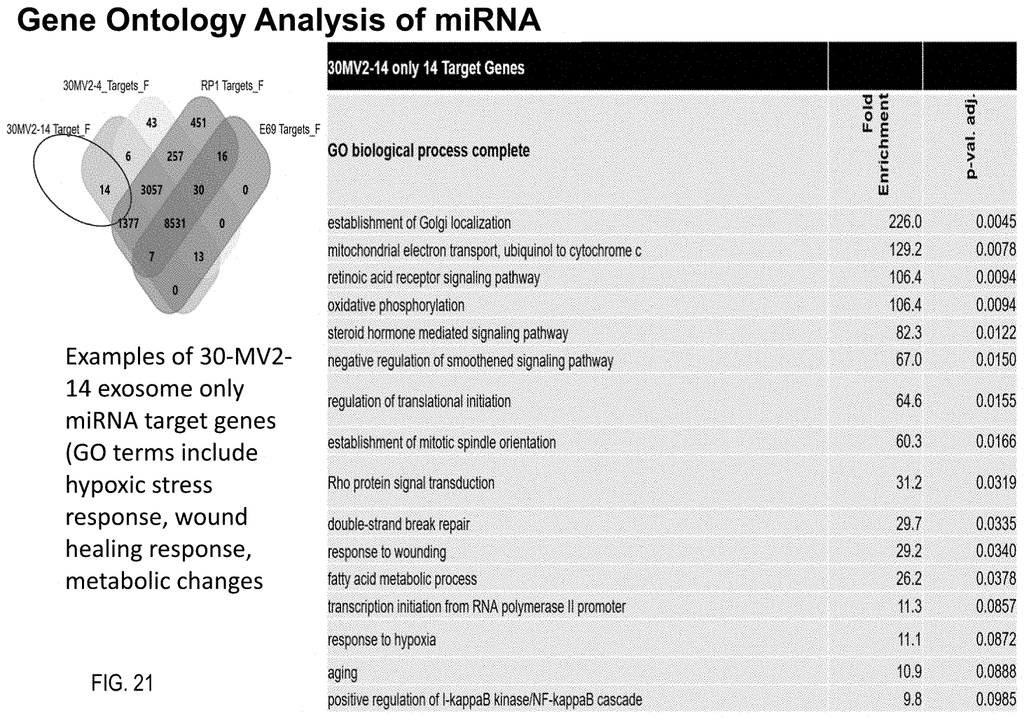

FIGS. 19-21 shows examples of RP1-MV2-8, 30-MV2-4, 30-MV2-14, exosome only miRNA target genes.

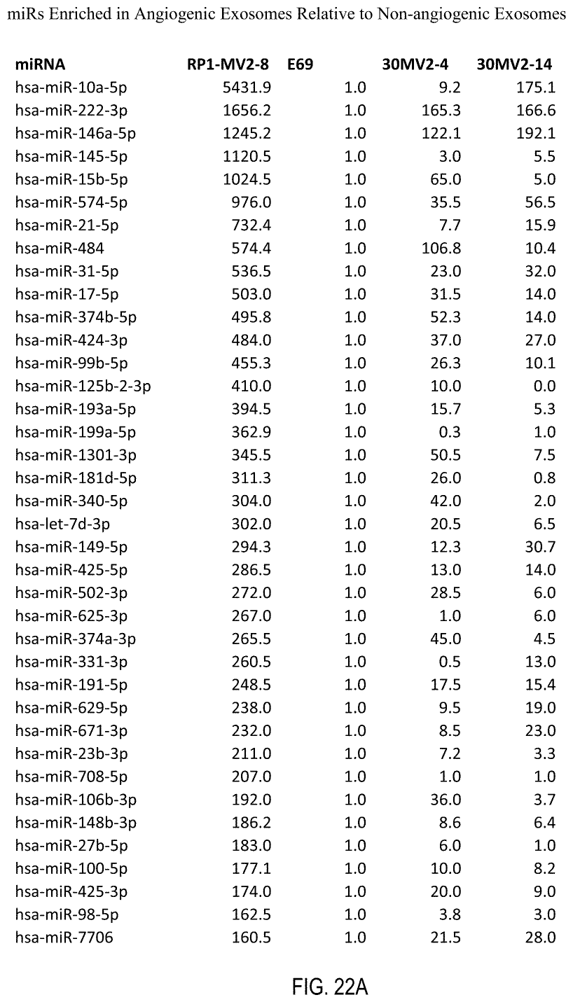

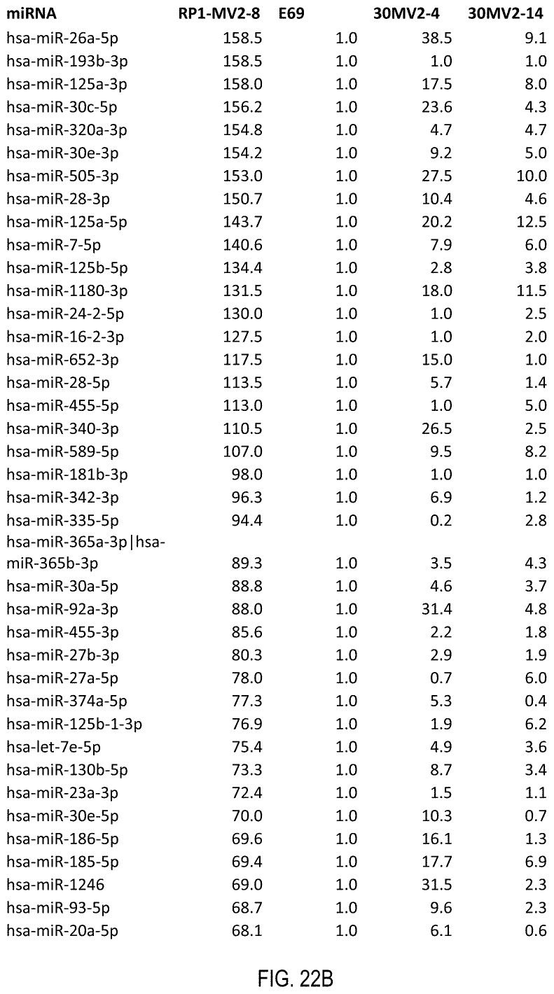

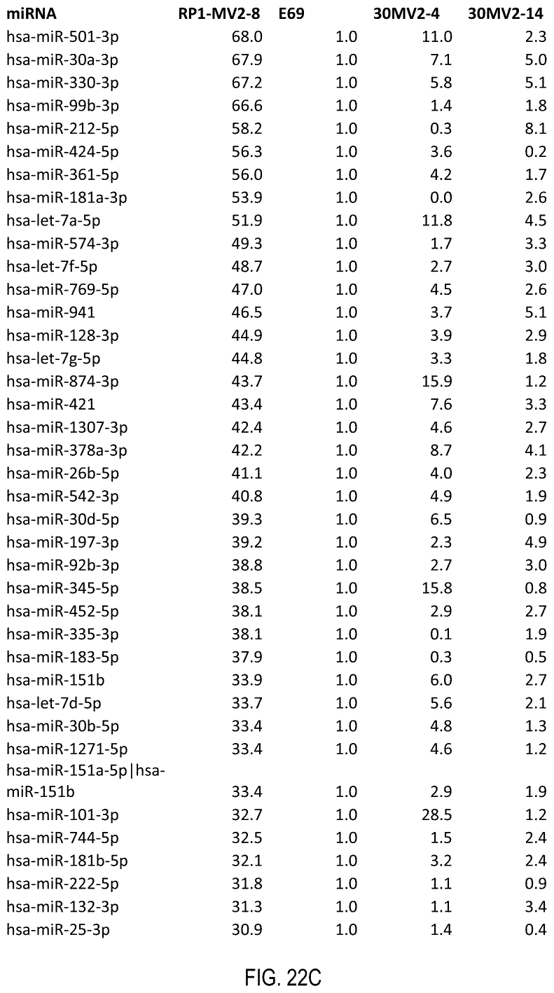

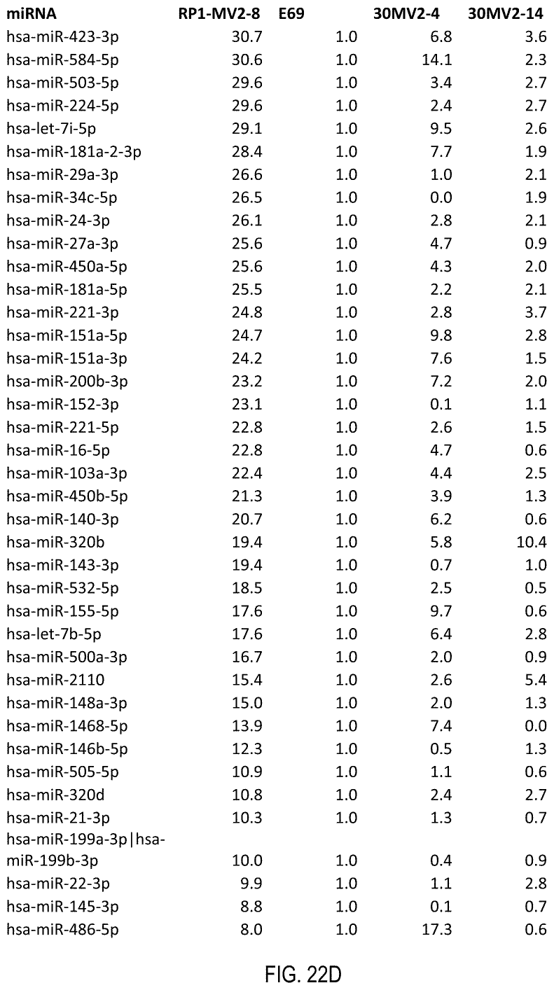

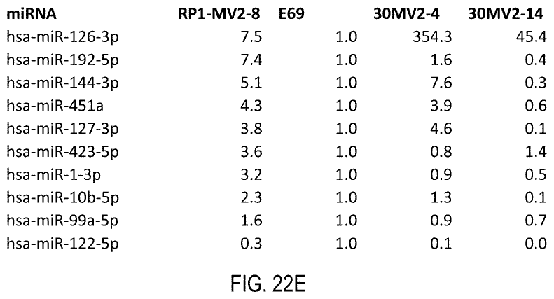

FIGS. 22A-E show miRNAs enriched in angiogenic exosomes relative to non-angiogenic exosomes.

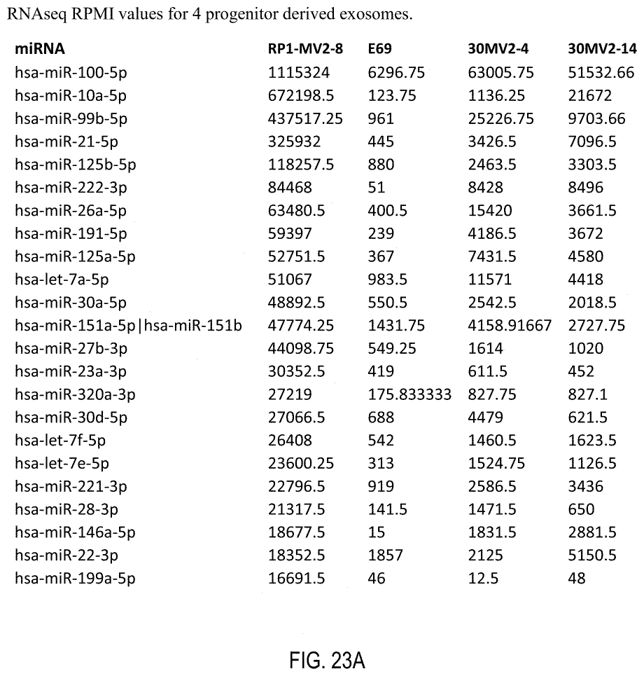

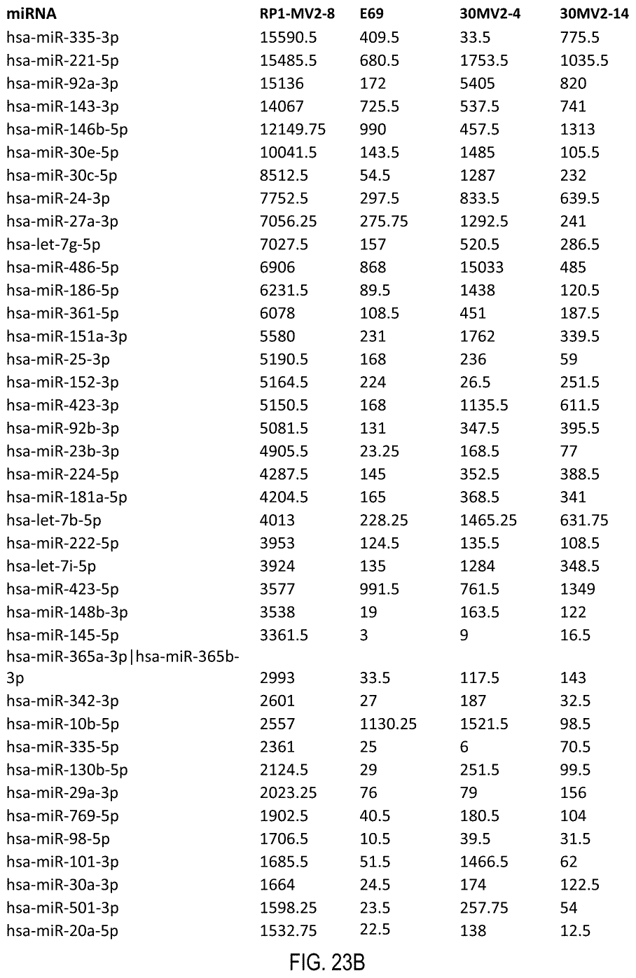

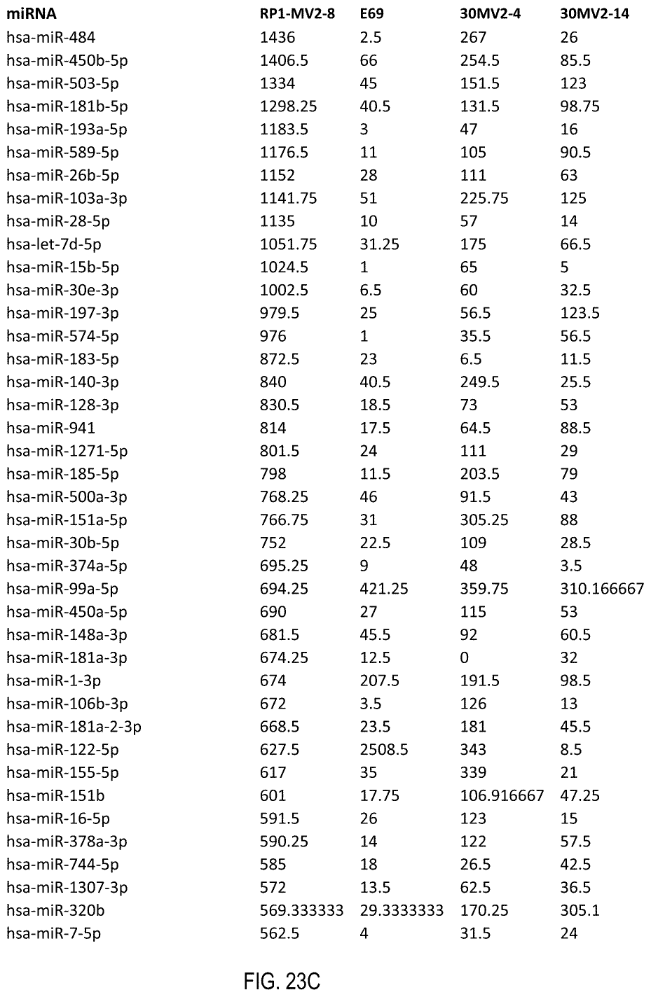

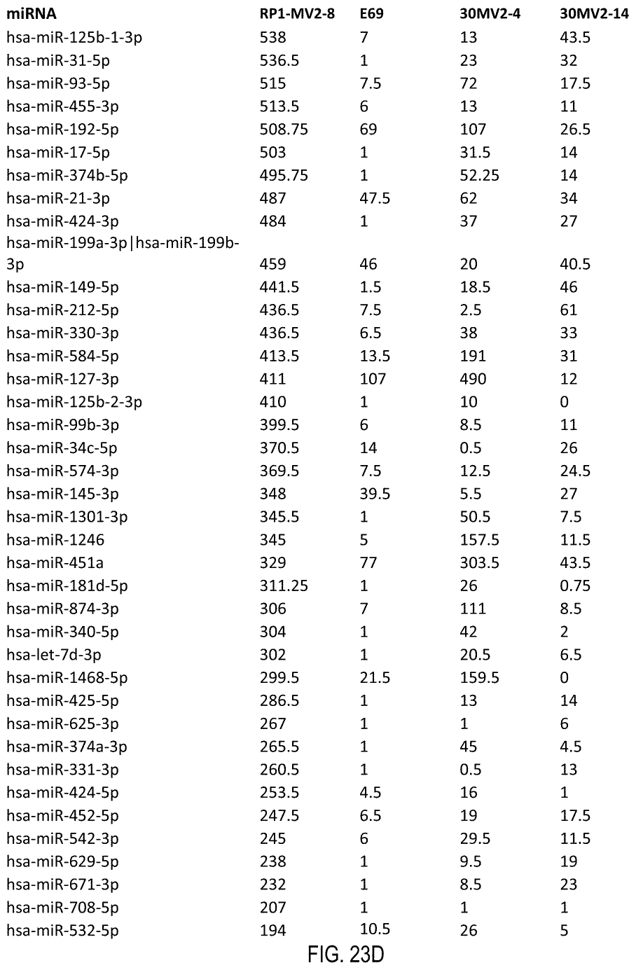

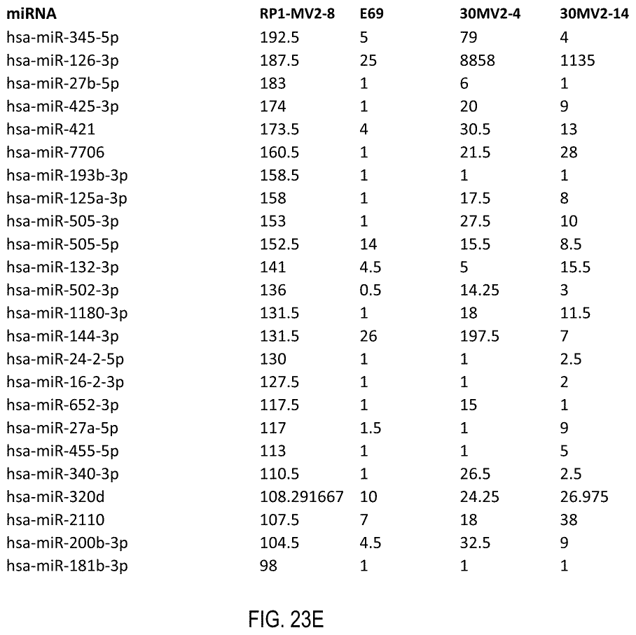

FIGS. 23A-E show RNAseq RPMI values for RP1-MV2-8, E69, 30MV2-4, and 30MV2-14 derived exosomes.

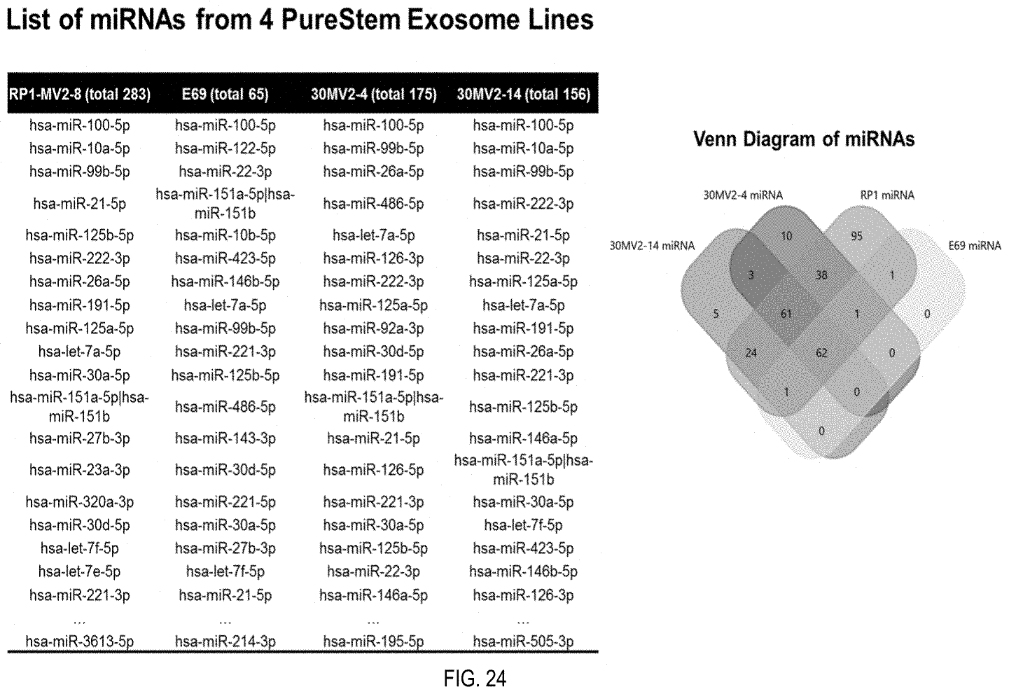

FIG. 24 shows lists of miRNAs from RP1-MV2-8, E69, 30MV2-4, and 30MV2-14 derived exosomes.

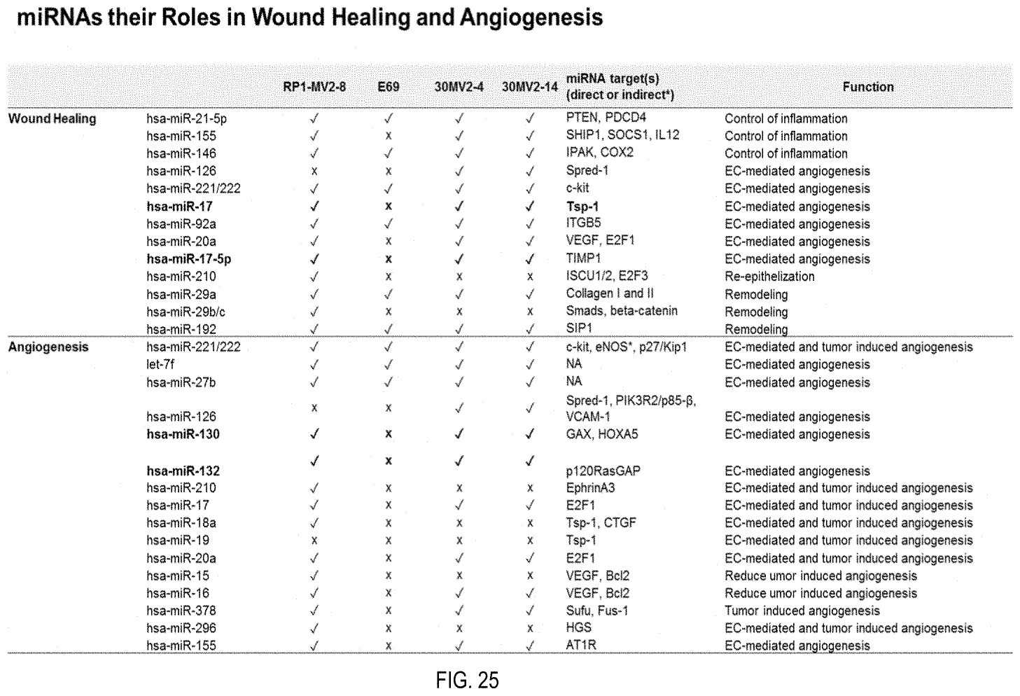

FIG. 25 shows the miRNAs and their roles in wound healing and angiogenesis.

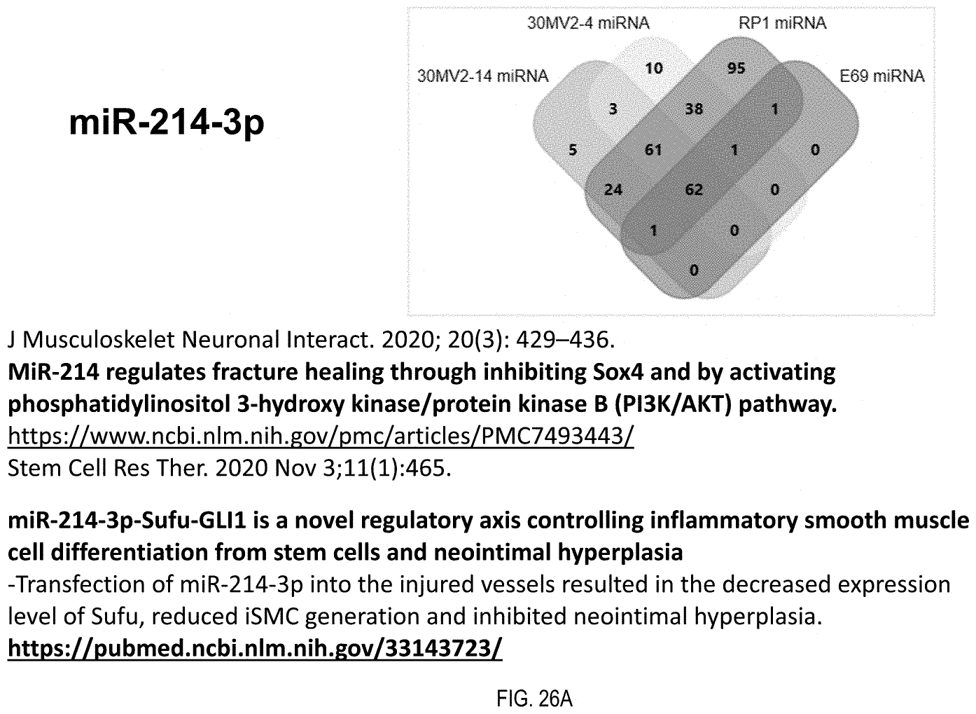

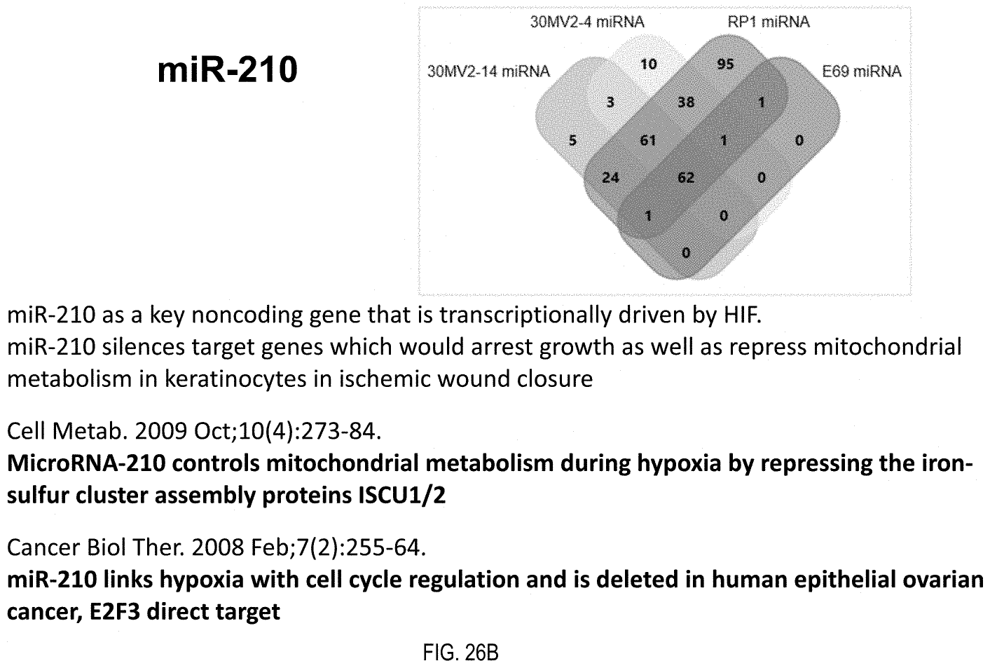

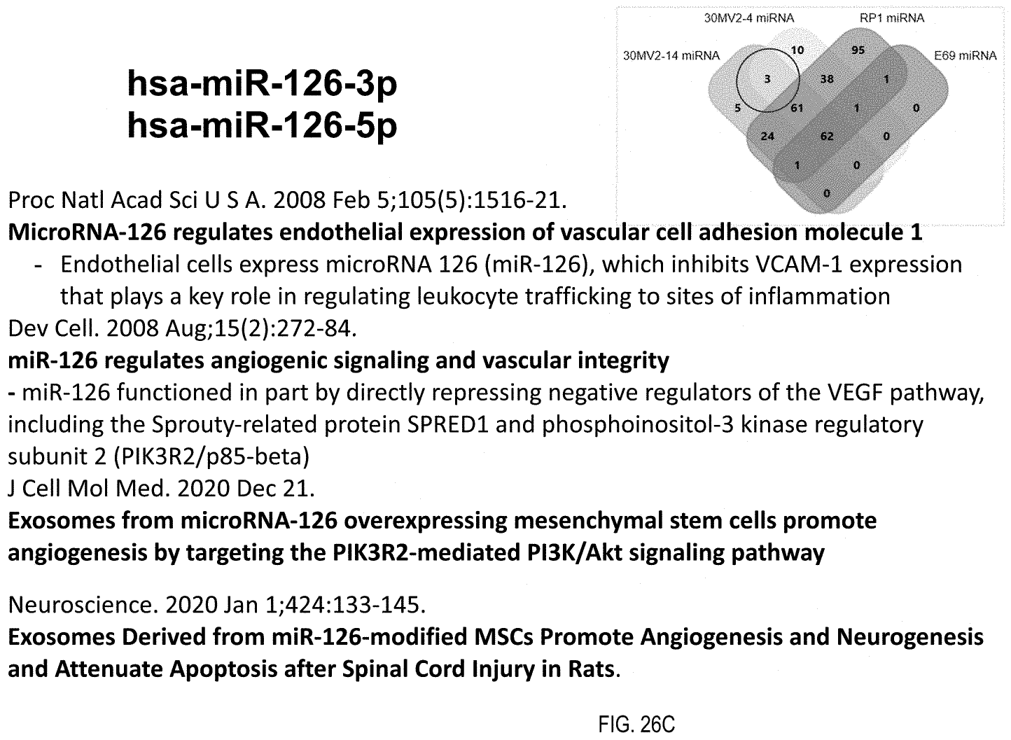

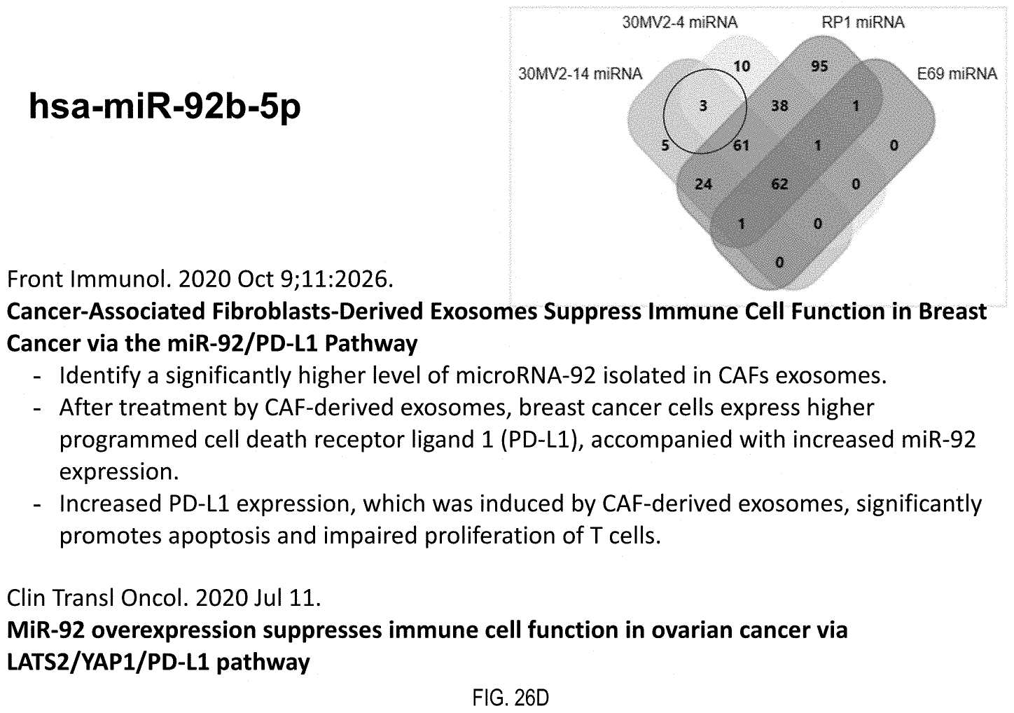

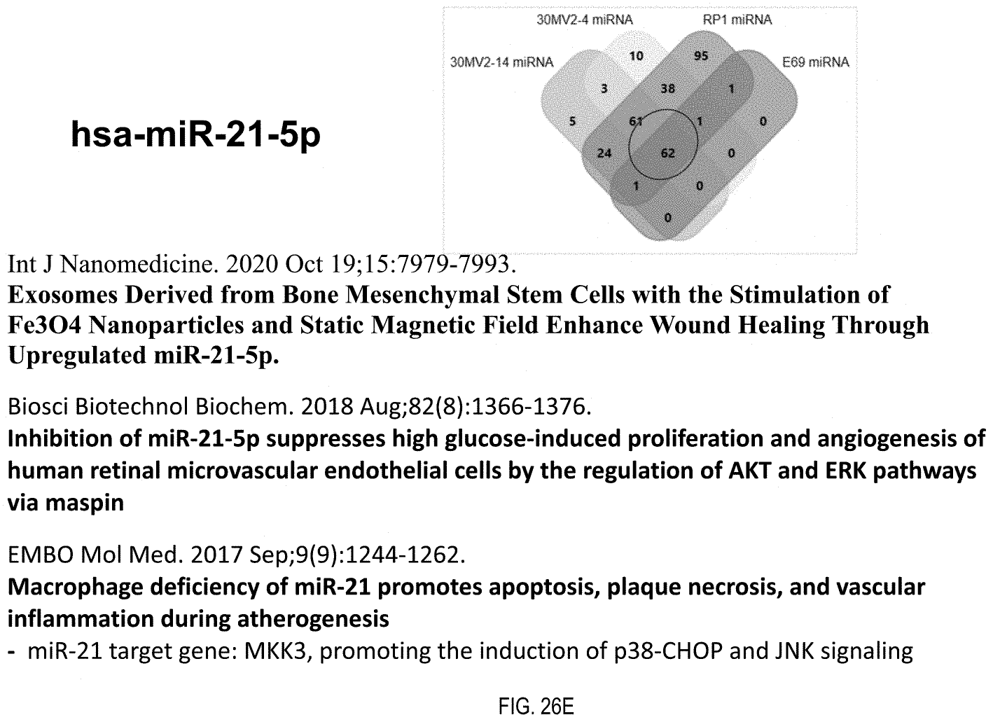

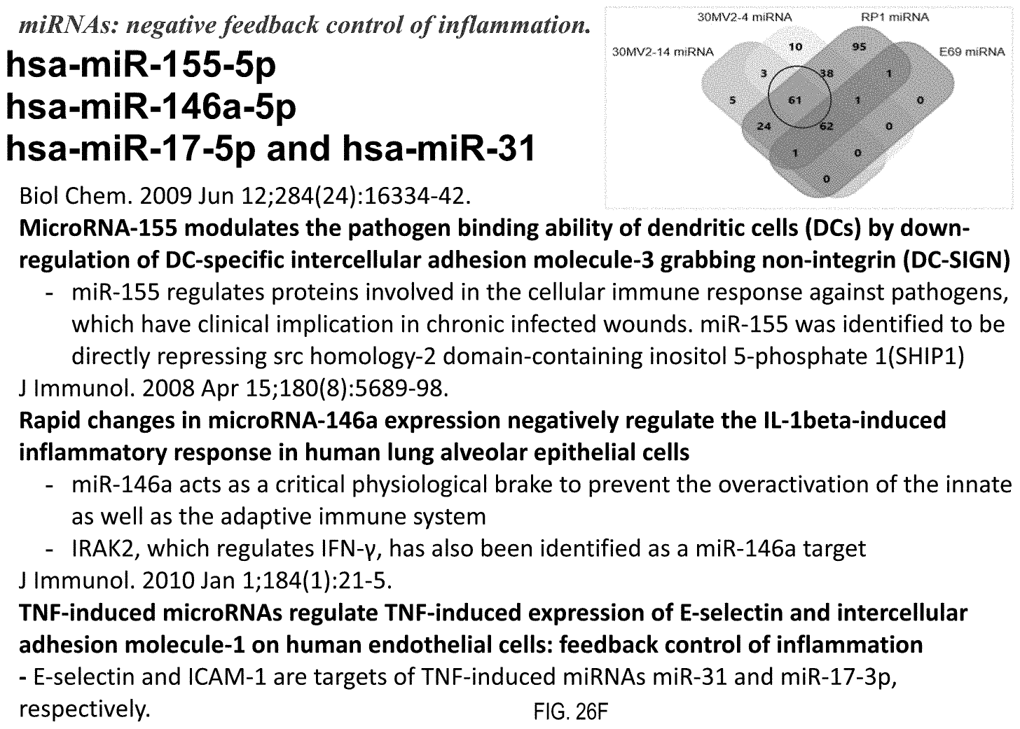

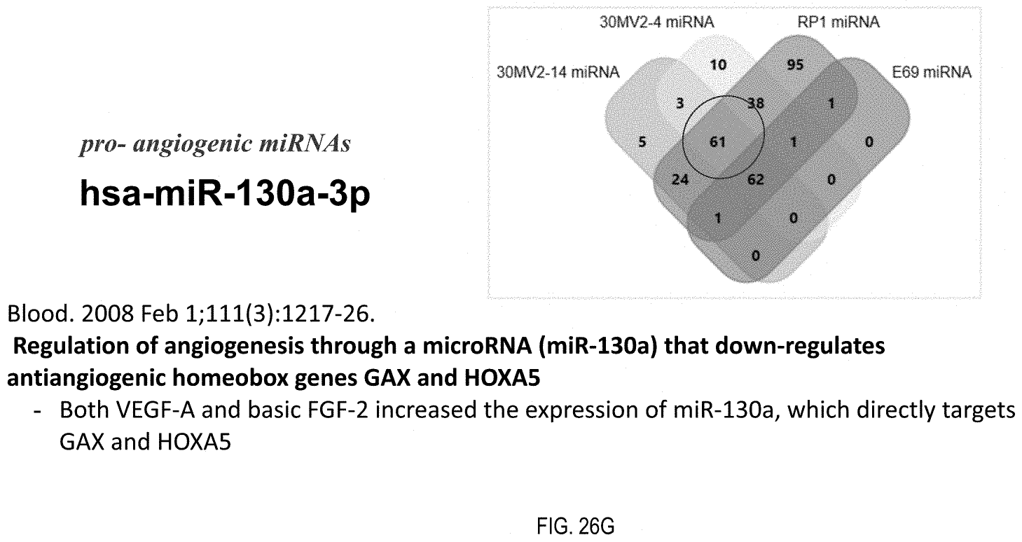

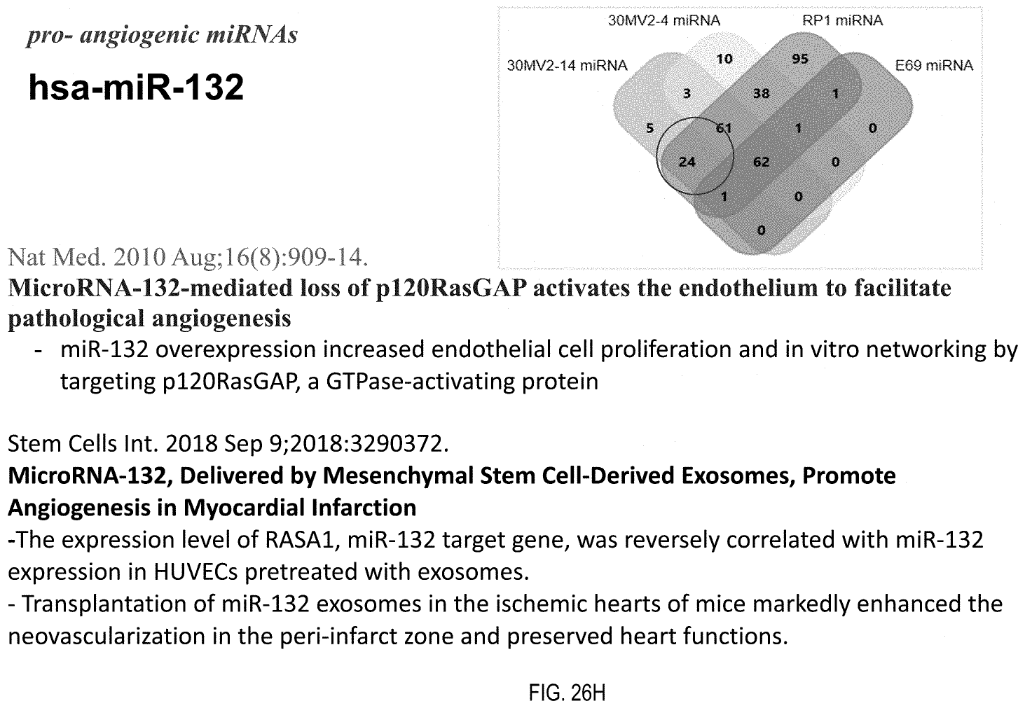

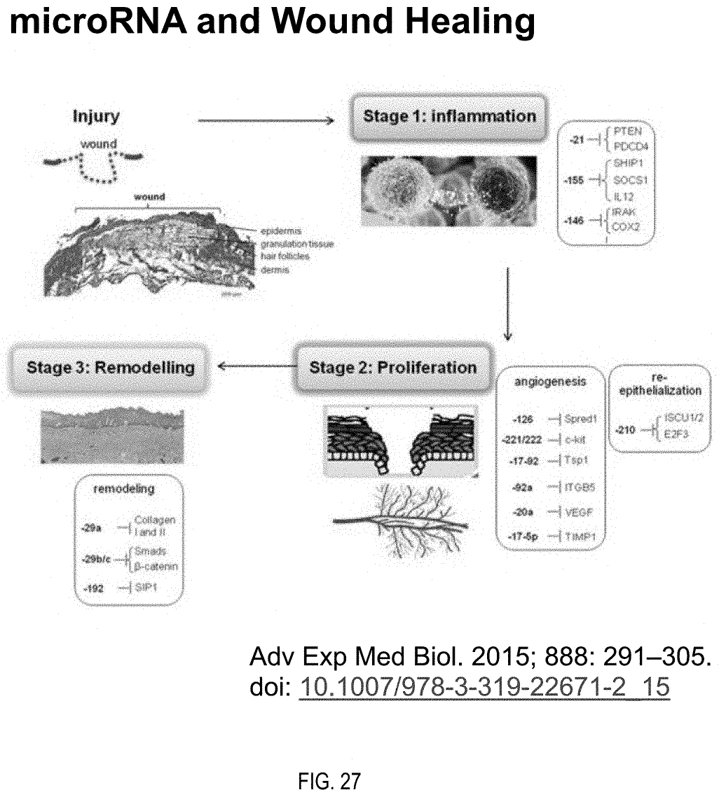

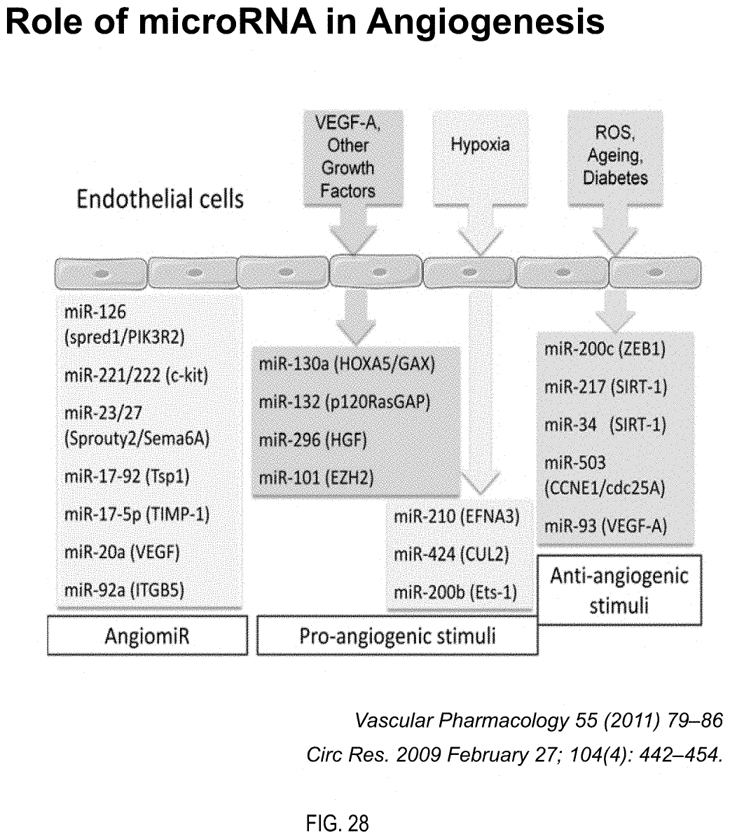

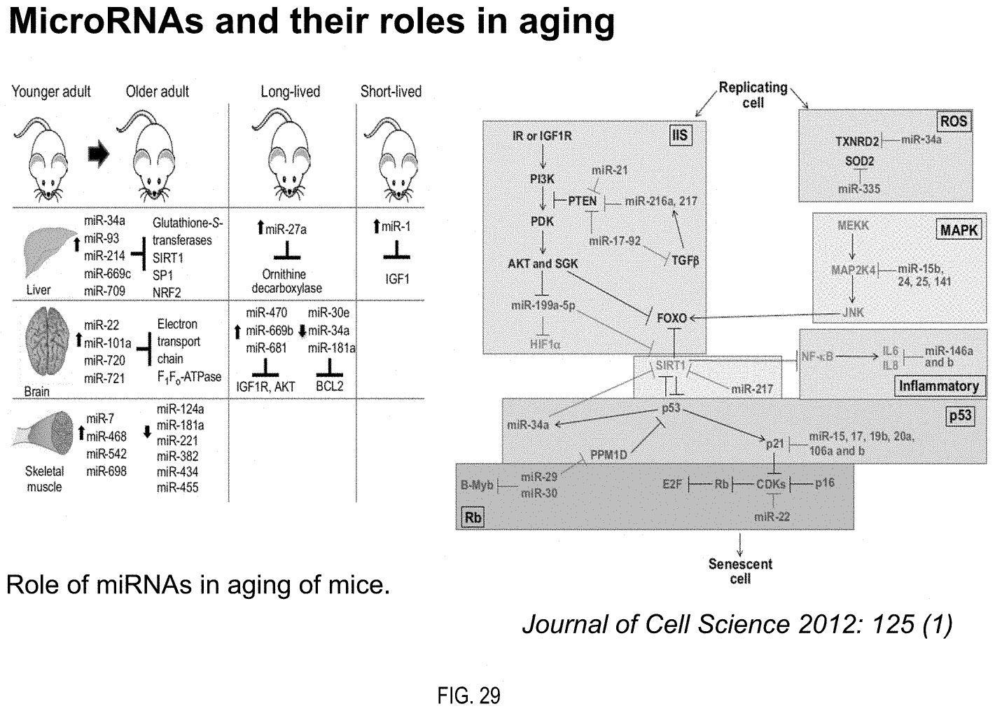

FIGS. 26A-H show functions of various miRNA.

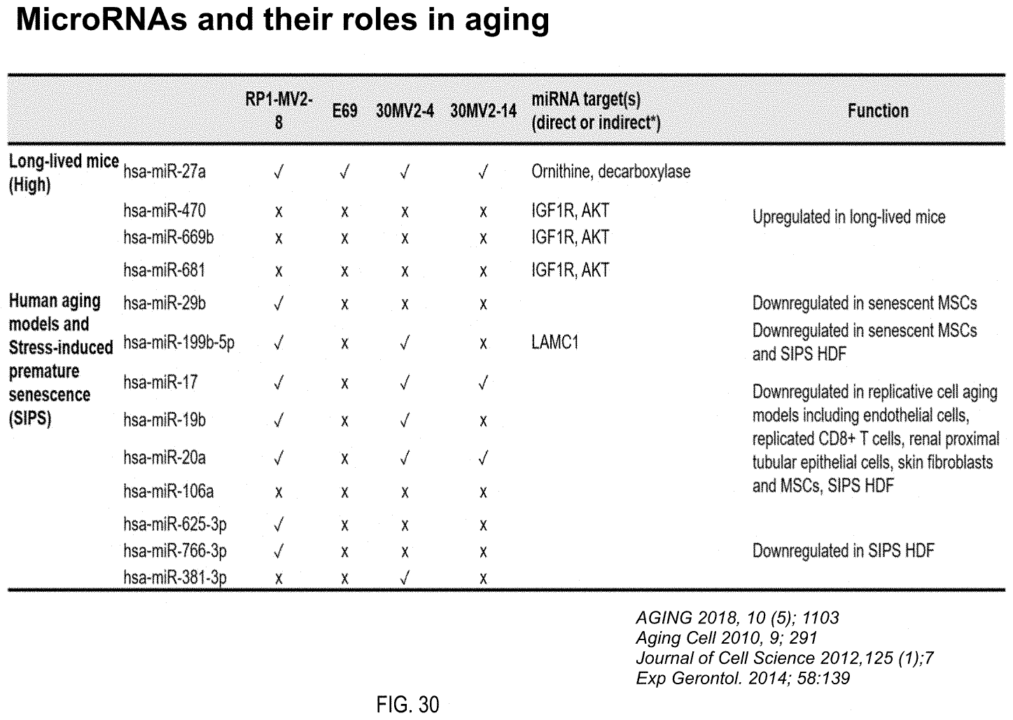

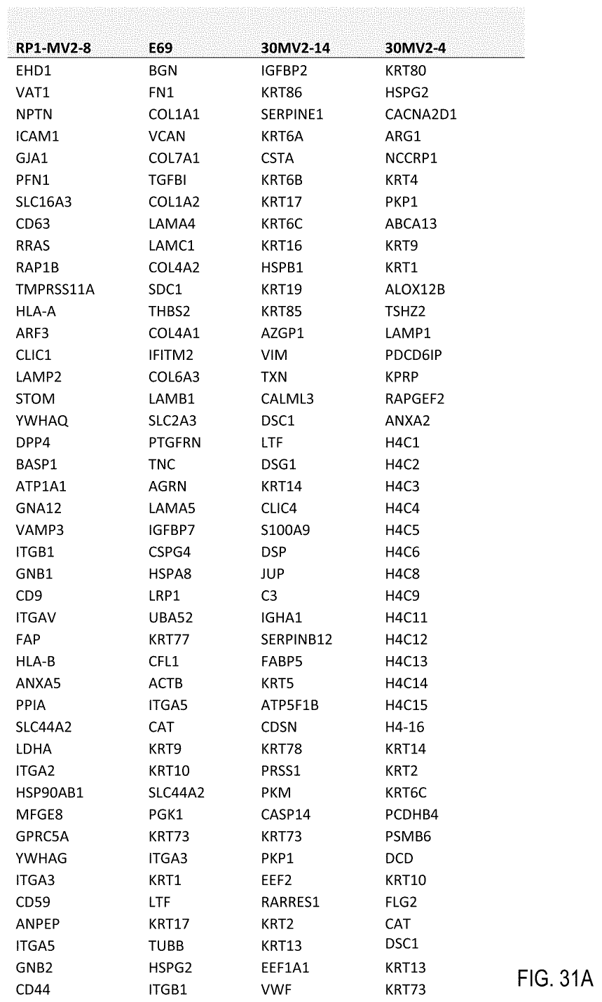

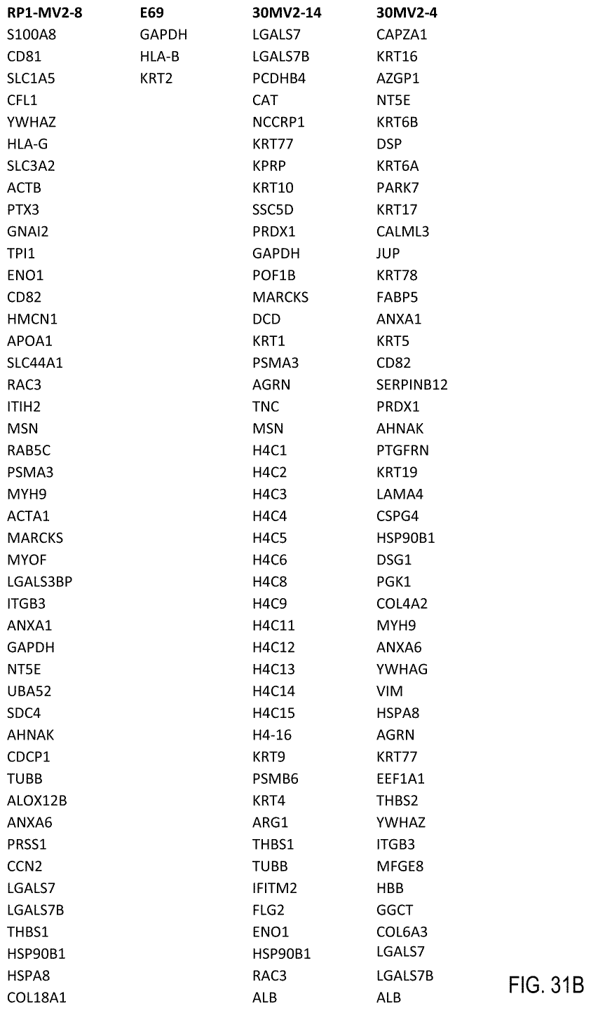

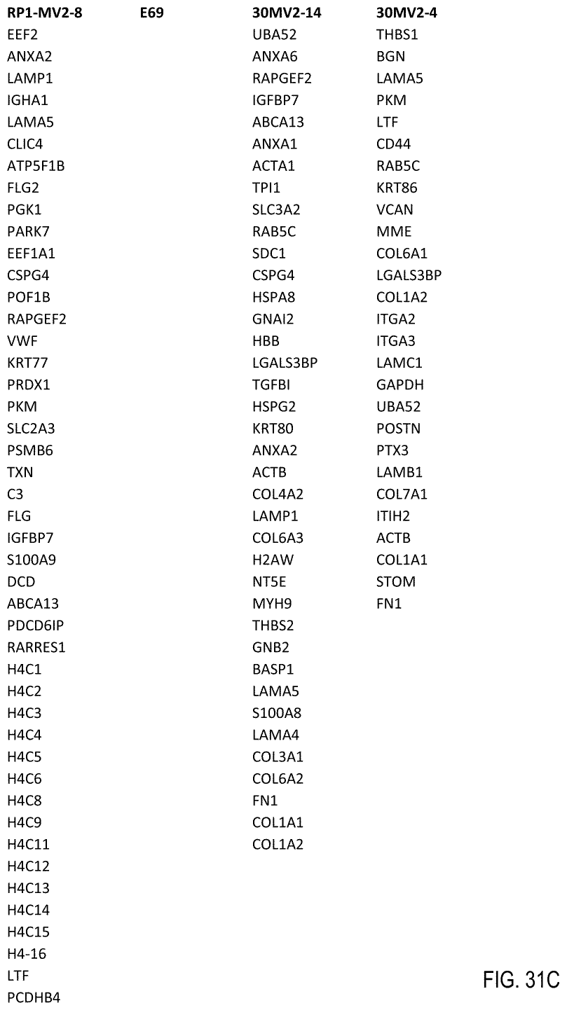

FIG. 30 show miRNAs having a role in aging.

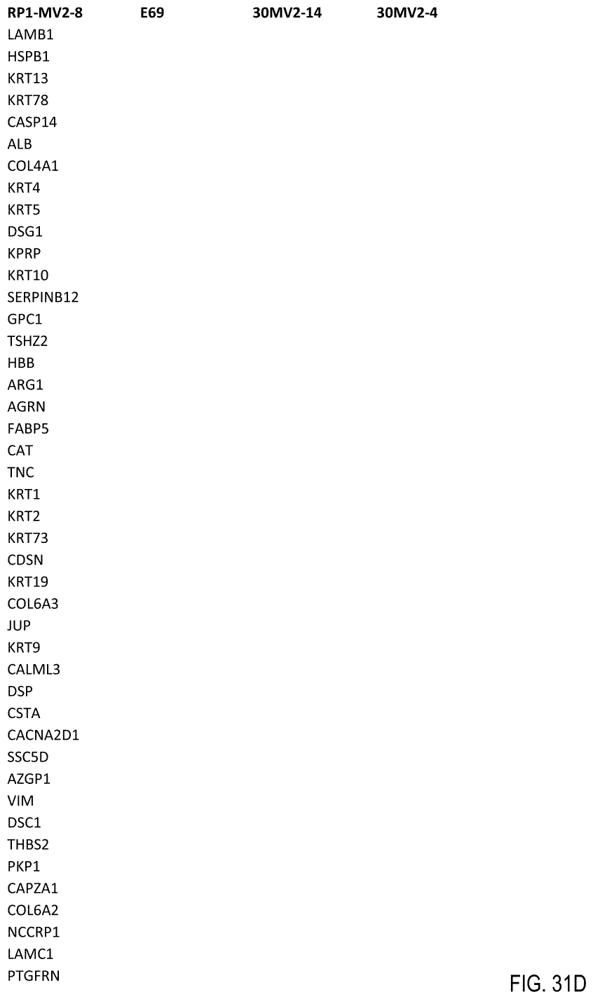

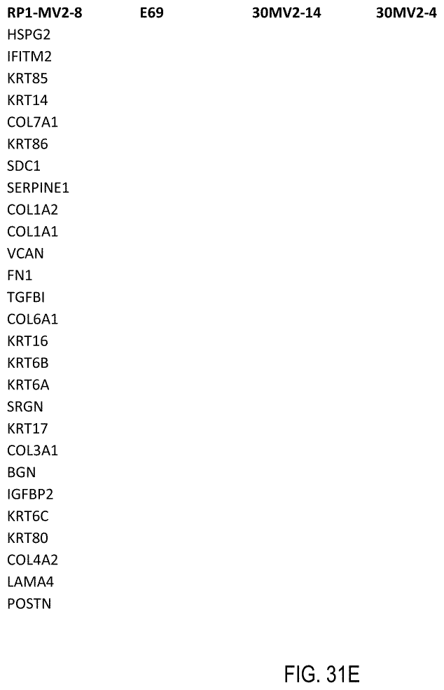

FIG. 31A-E shows total protein abundance in RP1-MV2-8, E69, 30MV2-14, and 30MV2-4.

In a preferred embodiment, the above data is used to select compositions and methods that employ exosomes providing beneficial utilities.

The above description of the disclosure is provided to enable a person skilled in the art to make or use the inventions described in the disclosure. Various modifications to the disclosure will be readily apparent to those skilled in the art, and the common principles defined herein may be applied to other variations without departing from the spirit or scope of the disclosure. Further, the above description in connection with the drawings describes examples and does not represent the only examples that may be implemented or that are within the scope of the claims.

1. An exosome loaded with one or more molecules to provide a therapeutic effect.

2. The exosome of claim 1, wherein the exosome is isolated from clonal progenitor cell line 30 MV2-14, 30-MV2-4, E69, or RPI-MV2-8.

3. The exosome of claim 1, wherein the exosome is capable of accelerating wound healing.

4. The exosome of claim 1, wherein the wound healing is measured by a migration assay and the percent of relative wound density is accelerated over that of a control without added exosomes.

5. The exosome of claim 1, wherein the exosome is capable of accelerating angiogenesis.

6. The exosome of claim 5, wherein the angiogenesis is observed by tube formation within 14 days.

7. The exosome of claim 1, wherein the exosome is capable of reducing the effects of aging.

8. The exosome of claim 1, wherein the exosome is capable of cardioprotection.

9. The exosome of claim 1, wherein the exosome is capable of neuroprotection.

10. The exosome of claim 1, wherein the exosome is capable of cardiac repair or regeneration.

11. The exosome of claim 1, wherein the exosome is capable of regulating immune activity.

12. The exosome of claim 1, wherein the exosome is capable of increasing vaccine outcome or vaccine potency.

13. The exosome of claim 1, wherein the exosome is loaded with miRNA.

14. The exosome of claim 13, wherein the miRNA is loaded via electroporation.

15. The exosome of claim 1, wherein the exosome is capable of providing epigenetic rejuvenation.

16. The exosome of claim 1, wherein the exosome is capable of modulating senolytic activity.

17. A method of preparing an exosome containing one or more molecules to provide a therapeutic effect.

18. The method of claim 17, wherein the exosome is isolated from clonal progenitor cell line 30 MV2-14, 30-MV2-4, E69, or RPI-MV2-8.

19. The method of claim 17, wherein the exosome is capable of accelerating wound healing.

20. The method of claim 17, wherein the wound healing is measured by a migration assay and the percent of relative wound density is accelerated over that of a control without added exosomes.

21. The method of claim 17, wherein the exosome is capable of accelerating angiogenesis.

22. The method of claim 17, wherein the angiogenesis is observed by tube formation within 14 days.

23. The method of claim 17, wherein the exosome is loaded with miRNA.

24. The method of claim 23, wherein the miRNA is loaded via electroporation.

25. The method of claim 17, wherein the exosome is capable of providing epigenetic rejuvenation.

26. The method of claim 17, wherein the exosome is capable of modulating senolytic activity.

27. The method of claim 17, wherein the exosome is capable of cardioprotection.

28. The method of claim 17, wherein the exosome is capable of neuroprotection.

29. The method of claim 17, wherein the exosome is capable of cardiac repair or regeneration.

30. The method of claim 17, wherein the exosome is capable of regulating immune activity.

31. The method of claim 17, wherein the exosome is capable of reducing the effects of aging.

32. The method of claim 17, wherein the exosome is capable of increasing vaccine outcome or vaccine potency.
